SEQUENCE LISTING (43 sequences)

SEQ ID NO: 1 - protein, 12 aa, Homo sapiens
ISSIKEKYPSHS

SEQ ID NO: 2 - DNA, 2979 bp, Homo sapiens
gctttgctcagccgccgcttttctctcccttctcactttctctcctccccacccagccctccttttcacctccaaaccctttagctgcgcgctgatcgcttctgtggctttgtttgggggtagggggagaatacgaatagacccgaggtgctttctcttggagctggagtccaatcccaatccctatctctatccctatccctgtgcttatccctgggcattagcccgggaccatggctggttcccgcagctgcctccggagcgcacctggggccatggtttgccgcgatcggagtccgctctaagtcggagtctgggggcgcagaagggggagcgcgccgagaaagtgccgggcaaacgcgcgccgcgcctggctgggcatcgcgagctgggcgaggagacggggaggggacagcccaaactatctcagcttcaccgccagtggtcacctacctgcagctatggagtcgccaaccaaggagattgaagaatttgagagcaactctctgaaatacctgcaaccggaacagatcgagaaaatctggcttcggctccgcgggctgaggaaatataagaaaacgtcccagagattacggtctttggtcaaacaattagagagaggggaagcttcagtggtagatcttaagaagaatttggaatatgcagccacagtgcttgaatctgtgtatattgatgaaacaaggagactcctggatacagaggatgagctcagtgacattcagtcagatgctgtgccttctgaggtccgagactggctggcctccaccttcacgcggcagatggggatgatgctcaggaggagcgacgagaagccccggttcaagagcatcgttcacgcagtgcaggctgggatatttgtggagagaatgtatagacggacatcaaacatggttggactgagctatccaccagctgttattgaggcattaaaggatgtggacaagtggtcctttgacgtcttttccctcaatgaggccagtggggatcatgcactgaaatttattttctatgaactactcacacgttatgatctgatcagccgtttcaagatccccatttctgcacttgtctcatttgtggaggccctggaagtgggatacagcaagcacaaaaatccttaccataacttaatgcacgctgccgatgttacacagacagtgcattacctcctctataagacaggagtggcgaactggctgacggagctggagatctttgctataatcttctcagctgccatccatgactacgagcataccggaaccaccaacaatttccacattcagactcggtctgatccagctattctgtataatgacagatctgtactggagaatcaccatttaagtgcagcttatcgccttctgcaagatgacgaggaaatgaatattttgattaacctctcaaaggatgactggagggagtttcgaaccttggtaattgaaatggtgatggccacagatatgtcttgtcacttccaacaaatcaaagcaatgaagactgctctgcagcagccagaagccattgaaaagccaaaagccttatcccttatgctgcatacagcagatattagccatccagcaaaagcatgggacctccatcatcgctggacaatgtcactcctggaggagttcttcagacagggtgacagagaagcagagctggggctgcctttttctcctctgtgtgaccgaaagtccactatggttgctcagtcacaagtaggtttcattgatttcatcgtggaacccaccttcactgtgcttacggacatgaccgagaagattgtgagtccattaatcgatgaaacctctcaaactggtgggacaggacagaggcgttcgagtttgaatagcatcagctcgtcagatgccaagcgatcaggtgtcaagacctctggttcagagggaagtgccccgatcaacaattctgtcatctccgttgactataagagctttaaagctacttggacggaagtggtgcacatcaatcgggagagatggagggccaaggtacccaaagaggagaaggccaagaaggaagcagaggaaaaggctcgcctggccgcagaggagcagcaaaaggaaatggaagccaaaagccaggctgaagaaggcgcatctggcaaagctgagaaaaagacgtctggagaaactaagaatcaagtcaatggaacacgggcaaacaaaagtgacaaccctcgtgggaaaaactccaaagccgagaagtcatcaggagaacagcaacagaatggtgacttcaaagatggtaaaaataagacagacaagaaggatcactctaacatcggaaatgattcaaagaaaacagatgattcacaagagtaaaaaagacctcatagacaataaaagaggctgccagtgtcttgcatcattctagctgagcttcttcattctccttcttctccttcttccacaaagacccatatctggagaaggtgtacaactttcaaacacaagccccccaccccctgacccttggccttccctcacaccatctccttccaggggatgaatctttgggggttggtttgaggtcttagaactctgggggatattcccctgagcaaaacaaacaacgtgagatttttactcaaacagaaacaaaacatgaaggggcatcctcaaaatcctttgctaatgacctggctttcaaggcatctgtctggcctgatgagaatggacatcctggatatgctgggagaggcctgaaaaaagccacacacacagtaattgccattttatgactgtcaatgccgttactttaaatgttgtcatttttgcactggctactgatgatacagccatgctgacattcatcaccgcaaagatgatgattccagtctctggttcctttcctgagtcaggaacatttgttttctccaatttcctttcagacttaaaattgttcttatgctttttttcccacttctgtaataca

SEQ ID NO: 3 - DNA, 4676 bp, Mus musculus
gtagggcatagggctgggtggccaagggctgcctgtgggggcaggtgtggtactcctgccctgctgacagtgttatcccctccctccccacacggggtgccagccctgtcactagccccaagccaacaaaagacttttctcacttgtcagttcttgcatgtcagcagggcttgctgttctcctcttgcctccaaatcctctggggagccgccggtagcttctttggctttgtttgaagggaaaaagaacaggaacagagtcgaggcgctttctcccggacacagtccttatccctgtccctggcagtagtaagcatcatggctggtttccacagctgcctccagcgcgcacctggggccggagtcccctgtgatccttgcaggctctgagccatctctggggacgctgcaggggaagtgacaaagtgactgaaagactcactgggctggacccagcaaggagaggaggctgggaagggactacacaaactctctccacctcaccctcagtgagaacctgcagctatggagtctccaaccaaggaaattgaagaattcgagagcaactctctgaaacacctgcaacctgagcagatcgagaaaatctggcttcggctccgcgggctgaggaaatataagaaaacgtcccagaggttacggtctttggtcaagcaattagaacgaggagaggcctctgtggtagaccttaagaagaatctggaatatgcagccacagtgcttgaatctgtatatattgatgaaacaaggcgactgctggatacagaggatgagcttagtgacattcaatcagatgctgtgccttcagaagttcgggattggctggcctccaccttcacacggcagatggggatgatgcttaggagaagtgatgagaagcccaggttcaagagcatcgtccatgcggtgcaagctgggatatttgtggaaagaatgtacagacggacatcaaacatggttgggctgagctatccaccggctgtaatcgatgcattgaaggatgtggatacgtggtccttcgatgtcttttccctcaatgaggccagtggagatcatgcactgaagttcattttctatgaattactcacacgttatgacctgatcagccgttttaagataccgatttctgcacttgtctcatttgtggaggccctcgaagtgggttacagcaagcacaaaaatccttaccataacctgatgcatgcagctgacgtcacacagactgtgcattacctcctttataagacaggagtagcaaactggctgacagagctggagatctttgcaataatcttttcggctgccatccatgactatgaacatactggaactacaaacaatttccacattcagactcggtcagatccagctatcctgtacaatgacagatctgtcctggagaaccaccacctgagtgcagcctaccgccttctgcaggaagacgaagagatgaatattctggttaacctctcaaaggatgactggagggagtttcgaactttggttattgagatggtaatggccacagatatgtcctgtcatttccagcaaatcaaagccatgaagacagccctgcagcaaccagaagcaattgagaagccgaaagccttatccctcatgttacacacagcagacatcagtcatcctgcgaaagcatgggacctgcaccaccgctggaccatgtctctcctggaggagttctttagacagggtgacagagaagcagagctggggctgccattttctcctctttgtgacagaaagtcaaccatggttgctcagtcacaagtgggttttattgacttcattgtggagcccaccttcactgtgctcacggacatgaccgagaagattgtgagtccattaattgacgaaagctcccagactggtgggacagggcagaggagatcaagtttgaacagcatcaactcatcagatgcaaagcgatccggtgtcaagagttctgggtcagatggaagtgctcccatcaacaattctgtcattcctgttgactataagagttttaaagccacttggactgaggtggtgcagatcaatcgggagcggtggcgagccaaggtacccaaagaagaaaaagccaagaaggaagctgaagagaaggctcgcctggctgctgaggaaaagcaaaaggaaatggaagccaaaagccaagctgaacaaggcacaaccagcaaaggcgagaaaaagacatcaggagaagccaaaagtcaagtcaatggaacacgcaagggtgataaccctcgtgggaagaactccaaaggagaaaaggcaggcgaaaagcagcagaacggtgacttgaaagacggtaaaaataaggcagacaagaaggatcactccaacaccggaaatgagtcaaagaaaacagatggtacaaagaagcgttctcatggctcacctgctccgagcactagttccacaagtcgcattaccttgccaggagactacggataaaacatcatccagaaacttccttggaagaaacctgaccatccccaaggtctgaagacaatgtggaaaatttatatttttttccccaaacaaaatctacatgtattgtaagttattgagtattaaacttcaattggtgagttttgctctccctaatccagctttttaaagacaagtccttttatatgtattttaaacctgcagacataagggaaaatgctgttaataacatatattaaaccctgataattttctgaggactttggtactagcatcttctttaacacttcaggtaactgaggcatgaataccttaatcacttgcccaaagtaattattggccactggcaagcctgttctcattcctgggagattcaagaaaaggctaaaggtaacttctgcacacttggagccaccttttcataaacttgccttatgtctggaggtttagatcatttctccacctgtgactttaaggctttatatctctggcatgaaggattctttcaagtaagtcccaaaatgaccactgacctggttatgcaactgactttcacttgcaatttcaagtgagagttggccattgttgctcactttctgtggactgaatcaaggcttatattttatgcctttctgtggcgccatagctgtacaattactaataatttcttgtcagaagcctctgaaatggtttagcacagacatgggcacgcagctaactgattagccatatgtttccattttgaatctaaataatcaagtgaaagaaacatccacatgttgatgaggaaaactaagtgaaaggctttccacaatgccacttgtcccagtggggaacatggactttgtgtgtgccaggacacacaagaggagaacataaaaatatgaaataaaatatgatcatctccctgagggaaggtttctttccttaaagcagaacagggtaattgaaggttgctgttgcttgaatatagaaatagttgaccttccagcccccttcccttcacttgtttgttaggagatgagactgaggcctatgcatcagtgcctttccctgttatagcgagacctacagatagaagccagagtcgcagcaactgtataaagcaatggttcactgcagtgtacacctgaagcttgtgatgggatggactcactggcatccttcttataaaagtcataggcagtgactctaggtatgatgacatcacaggagtaagggagaaatgctggtttctgttaacaccaacaacattgccagcaagtgcatagttaaagtgagtcatttgtttttttaaaggcaacacctgtgaaggttactgagtgtggcacaccgtagtttgttcactcatttaagcatttctctgcaccagctactatcaatattggttacatcagggaatgcaaaaatgtgtgtgcacattatccctacccagggatgtagatgtgaagggctgaagagggtatggaataaagaagtgcataaaacaagcaaccacccccacagagagtgactcaagcatttctatgtctcagtgtcaggaatgcagggtctcaggaatcaggatgggtcttgtcattttcatagcctccccacaatctactaattacatatagattagaagtcaggaacgaagctccatctcctcgttggctggctgagcattctccccatcagtatctctatctgagcattctccccatcagtatctctatctgagcattctccccatcagtatctctagcatctttttaggtccttctcagagtcattgagtgaacgtccatctttttacttgcttagttcaaaatgtgttttgtgtagtcttccttgcttagaataccacatcattttaaaacgtggtttccttgagaagttacgtggtatgttatctgacaaatgcaaagaaatagaatctcttcattatgtggtgagatcattcaggtgtaaatagtgtttggggaatttgataaatttgcaatagtgttataagaaaatcaagagaaaagactccatgataaaacatcctggtcttccatgctcatttatcatgtaatgattgttaccaagtacctactttttattcactgcaaagaaccagcttgtatattctccctggtatacaacctcttgaagttttcataacatccacggaaagaagttaccatttccccacatcatagtatagtt

SEQ ID NO: 4 - DNA, 3186 bp, Rattus norvegicus
aacacacctagaagaagcacgacttcagtccccgctattccctcggggtcccccaacctcctccagcctccagagagcggagcgcgaaggggcgcggcagcccgggccagcccacagctacacatagccttgtcagtccccctgccgtcgcagctgcagcgggctcgggatggggcagtggcaggggcgcaggggcttcactcggagccttggttattagctcccaggtctgggtgcctgcagccggcgtgagcagcttgtacctagcgcgggtctgcagtgacagtgcagcggagggaggctgggcgcgggctcaagccaggacccgggacacacgagtcatgacggacaccagccacaagaaggagggcttcaagaaatgtaggagcgccactttcagcatcgacgggtacagcttcaccatcgttgcaaatgaagcaggagataagaatgccagaccccttgcacgcttctcccgatctaagtcacagaactgtctgtggaactccctcattgatggcctcactgggaatgttaaggagaagccacggccaacaattgtccaggacacccgacccccagaagaaattctggcagatgaattgccacaactggatagcccagaagctttggtgaagacatccttcaggttacggtctttggtcaagcaattagagcgaggagaggcctctgtggtagatcttaagaagaatctggaatatgcagccacagtgcttgagtctgtatatattgatgaaacaaggcgcctgctggatacagaggatgaacttagtgacattcagtcagatgctgtgccttctgaagttcgggattggctggcctccaccttcactcggcagatggggatgatgcttaggagaagcgatgagaagcccaggttcaagagcattgtccatgcagtgcaagctgggatatttgtggagagaatgtatagacggacatcaaacatggttgggttgagctatccaccggctgtaatcgatgcactgaaggatgtggacacatggtccttcgatgtcttttccctcaatgaggccagtggagatcatgctttgaagttcattttctatgaattactcacacgttatgacttgatcagccgttttaagatacccatttctgcacttgtctcatttgtggaggcccttgaagttggttacagcaagcacaaaaatccttaccataacctgatgcatgccgccgatgtcacacagactgtgcattacctcctttataagacgggagtagcaaactggctgacggagctggagatctttgcaataatcttctcggctgccatccatgactatgaacatactggaactaccaacaatttccacattcagactcggtcggatccagctatcctgtacaatgacagatctgtactggagaaccaccacttgagtgcagcctaccgtcttctccaggaagatgaagagatgaatattctggtcaacctctcaaaggatgactggagggagtttcgaactttggtaatcgagatggtaatggccacagatatgtcctgtcatttccaacaaatcaaagccatgaagacagccctgcagcaaccagaagcaattgaaaagccgaaagccttatccctcatgctgcacacagcagacatcagtcatcctgcgaaagcgtgggacttgcaccaccgctggaccatgtctctcctggaggagttctttagacagggtgacagagaagctgagctggggctgccattttctcctctgtgtgaccgaaagtcaaccatggttgctcagtcacaagtgggttttattgacttcattgtggaacctaccttcactgtgctcacggatatgactgagaagattgtgagtccattaatcgatgaaacctcccagactggcgggacaggccagaggcgatcaagtttgaacagcatcaactcatccgatgcaaagcgatcaggtgtcaagagttctgggtcagaaggaagtgctcccatcaacaattctgtcatccctgttgactacaagagttttaaagccacttggaccgaggtggtgcagatcaatcgggagcggtggcgagccaaggttcccaaagaagagaaagccaagaaggaagctgaagagaaggctcgcctagctgctgaggaaaagcaaaaggaaatggaagccaaaagccaagctgaacaaggcacaaccagcaaagctgaaaaaaagacatcaggagaaacaaaaggtcaagtcaacggaacacgcacaagcaagggtgacaaccctcgtgggaagaactccaaaggcgacaaggcaggcgaaaagcagcagaatggtgacttgaaagatggtaaaaataaggcagacaagaaggatcactccaacaccggaaatgagtcaaagaaagcagatggtaccaagaagcgttctcatggctcacctgctccaagcactagttccacaagtcgccttaccttgccagtcatcaagcctcctttgcgtcactttaagcgtcctgcttatgcatctagttcctacgcaccttccgttccaaagaaaactgatgatcatcccgtaaggtacaagatgctggatcagcggatcaagataaaaaagatccagaacatctctcatcactggaacaaaaagtagggcaagtttaagaactgagggagtgggggagggccgaccgacctgcctgcctacctctcagcacctactacacacaccaggacacacccccgaagtcatgcagctggcactactgtggctgaccctgagagtcaaagtgaaatttgcctgagatccctcttggtcattttgttatagatgcaccagtcactacatttggagtcaatcaagactccagcaagaagctagtaaatggtagacttatttgattttactttgtaggcctttaaaagtcttaagttgcaatcaaattaaaaatgatatatcttatcctaaaaaataaaattcttcccactgccctgttttcatccccaagagatgatttacctttacatgaacactgccttaaaactgtcaggggtacgaaatgtacttttaaaaataacttttccaattaaaatgatgttattttgtgaaattaaaatataaatataccattttttctttcattttcccttcttccaaacctttacatatacatttgttctctctcaaaagc

SEQ ID NO: 5 - DNA, 1485 bp, Homo sapiens
gcacaaccccagcgcgagtgccagagcccagccggcgcggagcgggagcggtgcaggctgaggtctccgagcggctcgccatggctggcccgcagcagcagcccccttacctgcacctggccgagctgacggcgtcccagttcctggaaatatggaagcactttgacgcagacggaaatgggtatattgaaggtaaagagctagaaaactttttccaagagctggagaaggcaaggaaaggctctggcatgatgtcaaagagtgacaactttggagaaaagatgaaggagttcatgcagaagtatgataaaaactcagatgggaaaatcgagatggcagagctggcgcagatcctgccaaccgaagagaacttccttctgtgcttcaggcagcacgtgggctccagcgccgagtttatggaggcttggcggaagtacgacacagacaggagtggctacatcgaagccaatgagctcaagggattcctgtcagacctgctgaagaaggcgaaccggccgtacgatgagcccaagctccaggaatacacccaaaccatactacggatgtttgacttgaacggggatggcaaattgggcctctcagagatgtcccgactcctgcctgtccaggaaaacttcctgcttaaatttcagggcatgaagctgacctcagaggagtttaacgcgatcttcacattttacgacaaggatagaagcggctacattgacgagcatgagctggatgcccttttgaaggatctgtacgagaaaaacaaaaaggaaatgaatattcaacagctcaccaactacagaaagagcgtcatgtccttggcagaggcagggaagctctaccgcaaggacctggagattgtgctctgcagcgagccccccatgtaaagtggggacgggggctgcttctccacctcccccaaaccctgcttctgctgccctgatgcgtctacccagactcagagaccgtgagcgccccgcccccacccctacagcctgcacacacctgcctgcagagcaggaaatgagagatagaggatgggcagctggggggctgtcctgagccccctgcacccacccctgcccaggcagtctttgctcagtggatcacacacatggaaggtgatgggggcatgggtggagggtccctaattctcttcgctgtgatgcatgagctccctcgctgtatgatttaggcttctatgtccaacagagtggactcttccctctcgctcccctctgccggtcccccatgccaccacccaccccaaacttccaggttccatccaccaccttgccaatggtgtagctgtcctctcagaactcctgtgtgtggaaggcacccgccctttccttgccttctttactcggcgtgctccttttctctttgggtttcttgtttaccaaagaagagtttacagacaataaaatggaaaggtcctgctgtggaaacttacccaaaaaaaaaaaaaaaaaa

SEQ ID NO: 6 - DNA, 1462 bp, Mus musculus
accagagccaagcggcaccgagtgacagcgcgctgagagagaggcttaagatctccggagcggctcgccatggctggcccgcagcagcagcccccttacctgcacctggccgagctgactgcatcccagttcctggaaatctggaagcactttgatgctgacggaaatgggtacattgaaggtaaagagctagagaacttcttccaggagctggagaaggcaaggaagggttctggcatgatgtccaagagcgacaattttggagagaagatgaaggagttcatgcagaagtatgataagaactcagatgggaaaattgagatggcggagctggcgcagatcctgccgaccgaagagaatttccttttgtgcttcaggcagcacgtgggctccagcgctgagtttatggaggcttggcggaagtatgacacagacagaagtggctacatcgaagccaatgagctcaagggattcctgtctgacctcctgaagaaggccaacaggccctatgatgaacctaagctccaggagtacacccagaccatactacggatgtttgacttaaatggagatggcaaattgggtctctcagagatgtctagactcttgcctgtacaagagaacttcctgctgaaatttcagggtatgaagctgacctcagaagagttcaatgccatcttcacattttatgacaaggatggaagcggctatattgatgagaatgaactggacgccctcctgaaggatctgtatgagaagaacaagaaggagatgaacatccaacagctcaccacctacaggaagagtgtcatgtccttggccgaggcagggaagctctacagaaaggacctggagattgtgctctgcagtgagccccccgtgtaaaggggtgaagggacaggggctgcttctgcgcctcccttgaaccccgcccccgctgtcctgactctcttgacactccttcccagacctccccaccccctgccacctgcacacaccagcctgtggatctggaaaggagagatggagagagggtggctggtagggttccttaggcctgatagacagttgtgcctgcgttgggtcacggttggtgggcggggctgcaggggaagcttctgtcgtctccgctgcgatgcatgagttccttcgctgtatgatttaggcttctgagtcccacagagtggactccttcctcttgctgcgccacccctccccctttctccccggtccaccgctgaccccggacttccaggttctgcccaccagcttgcttaatgatctagctgtcttctcagagctactgtggagggtgactgccctctccttgtgttcttgacgtgtgtgttccttttctctttggcttcccctgtcttcttgtttaccaaagaagagtttacagagaataaagtggaaatgttctgtcgcggaaaaaaaaaaaaaaaaaaaaaaaaaaaaaaaa

SEQ ID NO: 7 - DNA, 1449 bp, Rattus norvegicus
gggcggcaccgagtgacagcgctgagaggcttaagatctccggagccgctcgccatggctggcccgcagcagcagcccccttacctgcacctggccgagctgactgcatcccagttcctggaaatctggaagcactttgatgctgacggaaatgggtacattgaaggtaaagagctagaaaacttcttccaggagctggagaaggcaaggaaaggttctggcatgatgtccaagagcgacaattttggagagaagatgaaggagttcatgcagaagtatgataagaactcagatgggaaaattgagatggcagagctggcgcagatcctgccaaccgaagagaatttccttttgtgcttcaggcagcacgtgggctccagcgctgagtttatggaggcttggcggaagtatgacacagacagaagtggctacatcgaagcaaatgagctcaagggatttctgtccgacctcctgaagaaggccaaccgtccctatgatgaacctaagctccaggagtacacccaaaccatactacgcatgtttgacttaaatggagacggcaaattgggtctctcagagatgtctagactcttgcctgtacaagaaaacttcctgctgaaatttcagggtatgaagctgacctcagaagagttcaatgccatcttcacattctatgacaaggatggaagcggctacatcgatgagaacgagctggacgccctcctgaaggatctgtatgagaagaacaagaaggagatgaatatccagcagctcaccacctacaggaagagcgtcatgtccttggccgaggcagggaaactctacagaaaggacctggagattgtgctttgcagtgagccccccatgtaaaggggtgaagggacaggggctgcttctgcacctcccctaaaccctgccccccactgccctgactctcttgaaactccttcccagacctctccacccctgcaacctgcacacaccagcctgtggggcaggaaaggagagatggaaagagggtggctggtagcattcctttgagctgcaccagcccaaccctggcctgacagacagctgtgcctgtggtgggtgggtcacacggttggtgggtaggggcacaggtggggcttccctagtctctttgctgtgatgcatgagctcctcgctgtatgattagcttccgagtccaacagagtgcactccttcctcttgctgcaccagcctccttctccccaggccccccactgacccccgcaattccaggttctgtccaccggcttgctaatgatataactgtcttctcagagctactgtggagggtcattgccctcttcttgtgttcttgacttgtgtgttccttttattttggggtttccctgtcttctcgtttaccaaagaagagtttacagagaataaagtggaaatgtcctgccgtgaaaaaaaaaaaaa

SEQ ID NO: 8 - DNA, 2722 bp, Homo sapiens
gagtctggccgcagtcgcggcagtggtggcttcccatccccaaaaggcgccctccgactccttgcgccgcactgctcgccgggccagtccggaaacgggtcgtggagctccgcaccactcccgctggttcccgaaggcagatcccttctcccgagagttgcgagaaactttcccttgtccccgacgctgcagcggctcgggtaccgtggcagccgcaggtttctgaaccccgggccacgctccccgcgcctcggcttcgcgctcgtgtagatcgttccctctctggttgcacgctggggatcccggacctcgattctgcgggcgagatgcccctgggacacatcatgaggctggacctggagaaaattgccctggagtacatcgtgccctgtctgcacgaggtgggcttctgctacctggacaacttcctgggcgaggtggtgggcgactgcgtcctggagcgcgtcaagcagctgcactgcaccggggccctgcgggacggccagctggcggggccgcgcgccggcgtctccaagcgacacctgcggggcgaccagatcacgtggatcgggggcaacgaggagggctgcgaggccatcagcttcctcctgtccctcatcgacaggctggtcctctactgcgggagccggctgggcaaatactacgtcaaggagaggtctaaggcaatggtggcttgctatccgggaaatggaacaggttatgttcgccacgtggacaaccccaacggtgatggtcgctgcatcacctgcatctactatctgaacaagaattgggatgccaagctacatggtgggatcctgcggatatttccagaggggaaatcattcatagcagatgtggagcccatttttgacagactcctgttcttctggtcagatcgtaggaacccacacgaagtgcagccctcttacgcaaccagatatgctatgactgtctggtactttgatgctgaagaaagggcagaagccaaaaagaaattcaggaatttaactaggaaaactgaatctgccctcactgaagactgaccgtgctctgaaatctgctggccttgttcattttagtaacggttcctgaattctcttaaattctttgagatccaaagatggcctcttcagtgacaacaatctccctgctacttcttgcatccttcacatccctgtcttgtgtgtggtacttcatgttttcttgccaagactgtgttgatcttcagatactctctttgccagatgaagttacttgctaactccagaaattcctgcagacatcctactcggccagcggtttacctgatagattcggtaatactatcaagagaagagcctaggagcacagcgagggaatgaaccttacttgcactttatgtatacttcctgatttgaaaggaggaggtttgaaaagaaaaaaatggaggtggtagatgccacagagaggcatcacggaagccttaacagcaggaaacagagaaatttgtgtcatctgaacaatttccagatgttcttaatccagggctgttggggtttctggagaattatcacaacctaatgacattaatacctctagaaagggctgctgtcatagtgaacaatttataagtgtcccatggggcagacactccttttttcccagtcctgcaacctggattttctgcctcagccccattttgctgaaaataatgactttctgaataaagatggcaacacaattttttctccattttcagttcttacctgggaacctaattccccagaagctaaaaaactagacattagttgttttggttgctttgttggaatggaatttaaatttaaatgaaaggaaaaatatatccctggtagttttgtgttaaccactgataactgtggaaagagctaggtctactgatatacaataaacatgtgtgcatcttgaacaatttgagaggggaggtggagttggaaatgtgggtgttcctgttttttttttttttttttttttagttttcctttttaatgagctcaccctttaacacaaaaaaagcaaggtgatgtattttaaaaaaggaagtggaaataaaaaaatctcaaagctatttgagttctcgtctgtccctagcagtctttcttcagctcacttggctctctagatccactgtggttggcagtatgaccagaatcatggaatttgctagaactgtggaagcttctactcctgcagtaagcacagatcgcactgcctcaataacttggtattgagcacgtattttgcaaaagctacttttcctagttttcagtattactttcatgttttaaaaatccctttaatttcttgcttgaaaatcccatgaacattaaagagccagaaatattttcctttgttatgtacggatatatatatatatagtcttccaagatagaagtttactttttcctcttctggttttggaaaatttccagataagacatgtcaccattaattctcaacgactgctctattttgttgtacggtaatagttatcaccttctaaattactatgtaatttattcacttattatgtttattgtcttgtatcctttctctggagtgtaagcacaatgaagacaggaattttgtatatttttaaccaatgcaacatactctcagcacctaaaatagtgccgggaacatagtaagggctcagtaaatacttgttgaataaactcagtctcctacattagcattctaa

SEQ ID NO: 9 - DNA, 2693 bp, Mus musculus
cctcccccggccacggccgccgcggcggtggctctgcatctccaaaaagggccctccgacttctcactgggccagtccggcgctgggtggcggagccctacactgctcccgccggtcccagagccgggatcccttctcttcagagtttcgagcaactttccccgcctctggagctaaagtggctcggggactgtgaccaccgcaggtgggagagtccccggcccggctctccgcatcttcgctgtgcgctcgcgctgggaaagccctgggtgcgtgcaccctgggggtccccggcctcggctctgtgggcgagatgcctctgggacacatcatgaggctggatctggagaagatcgccctggagtacatcgtgccctgtctgcacgaggtcggcttctgctacctggacaacttcctaggcgaggtggtgggcgactgcgtgctggagcgagtcaagcagctgcactacaacggggccctgcgtgacggccagctggccgggccgtgcgccggcgtctccaagcggcacctgcgaggcgaccagatcacgtggatcgggggcaacgaggagggctgcgaggccatcaacttcctcctgtccctcatcgaccggctggtcctgtactgcgggagccggctgggcaaatactatgtcaaggagcggtccaaggcaatggtggcttgctatccaggaaatgggacaggttatgttcgccatgtggacaaccccaatggtgatggccgctgtatcacctgtatctactacctgaataagaattgggacgccaagttacacggaggggtcctgcggatatttccggaagggaaatcgtttgtagcagacgtggagcccatttttgacagacttctgttcttctggtcagaccgcaggaatccacatgaagtccagccctcctatgccaccaggtacgctatgactgtctggtacttcgatgctgaagaaagggcagaagccaaaaagaaattcaggaatttaactagaaaaactgaatctgctcttgctaaagactgactgtggtgtgcaatccgctggccgcattcgtgtagtaacagttccggaaatgttaagtgtcaagatccaaagaatccatacaatcccctctgcccacgtcgttcagctccccaccttgtttgtggtacttcatgttttcttgccgggactgcggtgaccttcagacactctctttgccagatgaactcatttgctaactccagaaatacctgcagacgacatcctagctggccagcggtttaaagatagatttgggaattcggctgttgagcccaggactgcttcttattcgcactttatgtatgcgtcctgatttgaagggaggaggttcgcaaagaaataaagttggtgacagatgccacgaaggggtaccctccaaaccctcacagcaagaaagagaaatatctgaacgatttccagatgttgttaaccagagtctagacagtgacgacgacctaatgacattatcacctctagaaagggctgctgcttagcaatcgatttatagatatcccacggtggatgctccattttcctggtttacgacccgtactttctgaaggtcattgctaagtcaatggctctcttgtggaagatgatcccagcatttattatgcactatgtttacttcttacttggggaacctaattatccagagaataaaattcgccaggtagtttgggttgtttttctggtattgaaatttgaagttaaatcagaggatagaaagcttcctggtaattcagcactggattctgctaactctatgggaagagcaaagcccaacttacatgattcgtccgagcatccccatcccgagcagttcagttcggggagggagatggggttggaagtgcagacagggtcctttcatccgtgtgtgttccctttaacggttcatcgtccataacaaagaacagtgtgctgtactcttaaaagagatggaaataaaaagacctcagagctattggagttcttccacatcatctgctctgccagcctcacttgagtcgtcttgatccttgactgcaactggctggtaggacggccagagttgtgggatttcccagaaccgtgggagggtctcctgctgcagggggcaggtgtcgcttcctcccgaactctgtacgaaacacctattttacgaaagttagccaccctcgtttctgctgctacttccccaactttcaaacctctttaatttcctagctagaatcccacaagcagcatagacctgaaagtgttttatttcgtcatgttcagacagatagacagacagactttccaagacagaagttgatacttttcctctaattgttttggaaagtgtccaggaagaactgtcatcccacccccacccccaacaccccacccccgcccccaacacccccacccgtgccccgtcccgtggctcagttttgttttatttagtatttcccaccttctaacttgcaatgtcatgtccttatttattttgtttattgtctctttagcctgtgatgcctagagtgttaagtacaacgaagacagggattttgtacatttttgacccatgcaatattcccagcgcctaacctaatgcctggcatatagcaagaaagggcttagtaaatacatgctcaataaactcagtctcctctgtaaaaaaaaaaaaaaaaaa

SEQ ID NO: 10 - DNA, 2825 bp, Rattus norvegicus
ggatccaggttttcaagtttgttaggggatttaatgcaaatcaatatgcaaacagagagcattgtaaaaaaaaaaatccagtcctttctccggtcacacacaggttagctaatgtggagctcgcccagatgctctgcagcaccagctcaaggggaagggccaggaggagggcagtcccgggggcgggaccatgacgttgaggtcaaggcggggcttcctctcgttcctccccgggctgcggccgccgcggcggtggctccgcatctccaaaaggggccctccgacttctcactgggccagtccggcgctgggtggcagaactctacactactcctgccggtctcagagcgggactcccttctcttcagagtttcaagcaactttccccgcctttgcagctaaagtggctcggggaccgtggctgccgcaggtggtagagcccccggcccggctctctgcatctccgctgtgcgttcgctctgggcaagccctcggtgcgtgcaccctgggggtcccccgcctcggctctgtgagcgagatgcctctgggacacatcatgaggctggatctggagaagatcgccctggagtacatcgtgccctgtctgcacgaggtcggtttctgctacctggacaacttcctaggcgaggtggtgggcgactgcgtactggagcgagtcaagcagctgcattacaacggggccctgcgtgacggccagctggcggggccccgcgccggcgtctccaagcggcacctgcgaggcgaccagatcacgtggatcgggggcaacgaggagggctgcgaggccatcaacttcctcctgtccctcatcgacaggctggtcctgtactgcgggagccgcctgggcaaatactatgtcaaggagcggtccaaggcgatggtggcttgctatccaggaaatggaacaggttatgttcgtcatgtggacaaccccaatggtgatggccgctgtatcacctgtatctactacctgaataagaattgggacgccaagttacatggaggggtcctgcggatatttccagaaggaaaatcatttgtagcagacgtggagcccatttttgatagactcctgttctcctggtcagaccgcaggaatccacatgaagttcagccctcctatgccaccaggtacgccatgactgtctggtacttcgatgctgaagaaagggcagaagccaaaaagaaattcaggaatctaactagaaaaactgaatctgctcttgctaaagactgacggtggtgtgaagtccgctgggcgcattcgtttagtgacagttcctgaaatgttaagtgtcaagatccaaagactctgtccaatcccctgcctaccgcctgcatcgtttggctccctaccttgtttgtggtacttcgtgttttcttgctgggaatgcgttgccctccagacactctctttgccggatgaactcatttgctaactccagaaacacctgcagacatcctagctggccagcggtttaacggatagatctgggaatggatagatatgggaatgcagctgttcagcccaggattgcctcgtgttcgcactttatgcatacttcctggtctgataggaggaggtttgcgaagaaataaactggtgacagatgccacaaagaggcacccttgaaaccctaacagcaagaaagagaaagatctgaactatttccagatgttgttaacccgagactggacgattacggacaacctgatgacattatcgcctctagaaagggctgctgtcatagcgatcgatttataagtgtcccacggtggatgctcctttttcctggtcctacaacctgttctttttgaaggtcattgctaaattgatgctctcttgtcgaagacgaccccagcctgtatgctccatttttacttcttacttggggatcctaattatccagagaataaaactcaccaggtagtttgggttgttttttctggtactgaaaatttgaagttaaatgagagggacaggaagcgtcctggtaatttagcttctgctaactctgtgggaagagcaaagcctagcggtatgcgataaccatgcatacggtgagcagttcgagagggagatgtggttggaagtgcagacagccccctttcatcatgttccctttagctgttcttcgttcatggctaagaacagcatgttgtactcttaaaagatatggaaataaaaagacctcaaagctatcgagttctcccacattgtctgctctgccagcctcgctttagccgtcttgatccttgagagcaactggttggaagtgcatccagtggtgtggaatttcctagaactgtgggaggtttctcctgcaggatgcaggtgccgcttcccccagaactctgtacgaaacacccattttacggaagttagccatcctagttttcgctgctacttccccatctttcgaacctctttaatttccttgttagaatcccacaagcagcacagacccgagagtgttttatttcatcgtgttcagccatgcagactttccaagcaaagaagttgatacttctttcttctttcgggaaagtgtccaggaagaactgtcattcctccccccatggctctgttttgttttatttcccaccttctaacttacaacgtcatttccttatttattatgtttattgtcttgtagcctatgatgtctagagtgttaaacacaacgaaggcagggatttgtacgtttttgatccatgcagcatgttcccagcacctaagctaacatacagtaagcaacggcttaccaaacacatgttcaataaactcagtctcctctgtaaaa

SEQ ID NO: 11 - DNA, 3572 bp, Homo sapiens
gaataattctgctacaaggctgatttcaaggacatgaattgttgacctcatcccaacatcagaacctcagatgttctaatttttgcaccattccaggcaagttgatcttataaggaaataaaattgaaccttaggggtctgatggaaattcactgtgacattcaaatcaagaaaacttgctaatgcccacagagccttttccccatgggccctgatggtagcctccagaaggtgcagcctcaggtggtgccctttcttctgtggcaagaataaactttgggtcttggattgcaataccacctgtggagaaaatggtatgcgagggaaagcgatcagcctcttgcccttgtttcttcctcttgaccgccaagttctactggatcctcacaatgatgcaaagaactcacagccaggagtatgcccattccatacgggtggatggggacattattttggggggtctcttccctgtccacgcaaagggagagagaggggtgccttgtggggagctgaagaaggaaaaggggattcacagactggaggccatgctttatgcaattgaccagattaacaaggaccctgatctcctttccaacatcactctgggtgtccgcatcctcgacacgtgctctagggacacctatgctttggagcagtctctaacattcgtgcaggcattaatagagaaagatgcttcggatgtgaagtgtgctaatggagatccacccattttcaccaagcccgacaagatttctggcgtcataggtgctgcagcaagctccgtgtccatcatggttgctaacattttaagactttttaagatacctcaaatcagctatgcatccacagccccagagctaagtgataacaccaggtatgactttttctctcgagtggttccgcctgactcctaccaagcccaagccatggtggacatcgtgacagcactgggatggaattatgtttcgacactggcttctgaggggaactatggtgagagcggtgtggaggccttcacccagatctcgagggagattggtggtgtttgcattgctcagtcacagaaaatcccacgtgaaccaagacctggagaatttgaaaaaattatcaaacgcctgctagaaacacctaatgctcgagcagtgattatgtttgccaatgaggatgacatcaggaggatattggaagcagcaaaaaaactaaaccaaagtgggcattttctctggattggctcagatagttggggatccaaaatagcacctgtctatcagcaagaggagattgcagaaggggctgtgacaattttgcccaaacgagcatcaattgatggatttgatcgatactttagaagccgaactcttgccaataatcgaagaaatgtgtggtttgcagaattctgggaggagaattttggctgcaagttaggatcacatgggaaaaggaacagtcatataaagaaatgcacagggctggagcgaattgctcgggattcatcttatgaacaggaaggaaaggtccaatttgtaattgatgctgtatattccatggcttacgccctgcacaatatgcacaaagatctctgccctggatacattggcctttgtccacgaatgagtaccattgatgggaaagagctacttggttatattcgggctgtaaattttaatggcagtgctggcactcctgtcacttttaatgaaaacggagatgctcctggacgttatgatatcttccagtatcaaataaccaacaaaagcacagagtacaaagtcatcggccactggaccaatcagcttcatctaaaagtggaagacatgcagtgggctcatagagaacatactcacccggcgtctgtctgcagcctgccgtgtaagccaggggagaggaagaaaacggtgaaaggggtcccttgctgctggcactgtgaacgctgtgaaggttacaactaccaggtggatgagctgtcctgtgaactttgccctctggatcagagacccaacatgaaccgcacaggctgccagcttatccccatcatcaaattggagtggcattctccctgggctgtggtgcctgtgtttgttgcaatattgggaatcatcgccaccacctttgtgatcgtgacctttgtccgctataatgacacacctatcgtgagggcttcaggacgcgaacttagttacgtgctcctaacggggatttttctctgttattcaatcacgtttttaatgattgcagcaccagatacaatcatatgctccttccgacgggtcttcctaggacttggcatgtgtttcagctatgcagcccttctgaccaaaacaaaccgtatccaccgaatatttgagcaggggaagaaatctgtcacagcgcccaagttcattagtccagcatctcagctggtgatcaccttcagcctcatctccgtccagctccttggagtgtttgtctggtttgttgtggatcccccccacatcatcattgactatggagagcagcggacactagatccagagaaggccaggggagtgctcaagtgtgacatttctgatctctcactcatttgttcacttggatacagtatcctcttgatggtcacttgtactgtttatgccattaaaacgagaggtgtcccagagactttcaatgaagccaaacctattggatttaccatgtataccacctgcatcatttggttagctttcatccccatcttttttggtacagcccagtcagcagaaaagatgtacatccagacaacaacacttactgtctccatgagtttaagtgcttcagtatctctgggcatgctctatatgcccaaggtttatattataatttttcatccagaacagaatgttcaaaaacgcaagaggagcttcaaggctgtggtgacagctgccaccatgcaaagcaaactgatccaaaaaggaaatgacagaccaaatggcgaggtgaaaagtgaactctgtgagagtcttgaaaccaacacttcctctaccaagacaacatatatcagttacagcaatcattcaatctgaaacagggaaatggcacaatctgaagagatgtggtatatgatcttaaatgatgaacatgagaccgcaaaaattcactcctggagatctccgtagactacaatcaatcaaatcaatagtcagtcttgtaaggaacaaaaattagccatgagccaaaagtatcaataaacggggagtgaagaaacccgttttatacaataaaaccaatgagtgtcaagctaaagtattgcttattcatgagcagttaaaacaaatcacaaaaggaaaactaatgttagctcgtgaaaaaaaatgctgttgaaataaataatgtctgatgttattcttgtatttttctgtgattgtgagaactcccgttcctgtcccacattgtttaacttgtataagacaatgagtctgtttcttgtaatggctgaccagattgaagccctgggttgtgctaaaaataaatgcaatgattgatgcatgcaattttttatacaaataatttatttctaataataaaggaatgttttgcaaatgttaaaaaaaaaaaaa

SEQ ID NO: 12 - DNA, 3693 bp, Mus musculus
gattgcctggcctgagtgacctcctggacctgccctggcggaatctgaacttgctcggccaagaagcccaggaagaattctgctacagggctgaattcaaggacatgaattgttgacctcatcccaacatcagaacctcagatagcattctaatttatttgcaagccaactttatgtgggaataaacttgaacggaggatctgttggaaattcactgtgaagttcaaatcaagaaaacttgctgaaatccctaaagccttcccccatgggccctgatggtagcctccagaaggtgcagcctcaggtggtcccccttcttctgtggcaagaataaactttgggtcgctgactgcaataccacctgcggagaaaatggtttgtgagggaaagcgctcaacctcttgcccttgtttcttccttttgactgccaagttctactggatcctcacaatgatgcaaagaactcacagccaggagtatgcgcattccatccgcctggatggggacatcattttggggggtctttttcctgttcatgccaagggagaaagaggggtgccttgtggggacctgaagaaggaaaagggcatccacagacttgaggccatgctttatgcaatcgaccagattaataaggaccccgatctcctctccaatatcactctgggtgtccggatccttgacacatgttccagggacacctatgctttggagcagtcactaaccttcgtgcaggcactgatagagaaagacgcgtctgacgtgaagtgtgctaatggagacccacccatattcaccaagcccgacaagatttctggtgtcataggtgctgcagcaagctccgtgtccatcatggtggctaacattttaagactttttaagatacctcagattagctatgcatctacagccccagagctaagtgacaacaccaggtatgatttcttttctcgggtggtcccgcctgactcctaccaagcccaagccatggtggacattgtgacagccctgggatggaattatgtgtcaacactggcttccgaggggaactatggagagagtggtgttgaggccttcactcagatctcaagggagattggtggtgtttgcattgctcaatcacagaaaatcccacgtgaaccaagacctggagaattcgaaaaaattatcaaacgcctgctggagacacccaacgctcgcgcagtgattatgtttgccaatgaggatgacatcaggaggatattggaagcagcaaaaaaattaaaccagagtgggcattttctatggattggctcagatagttggggatccaaaatagcacctgtctatcagcaggaggagatcgccgaaggagctgtgacaattttgcccaaaagagcatcaattgatgggtttgaccgatactttagaagccgaactcttgccaataatcgaagaaatgtgtggtttgcagaattttgggaggagaattttggatgcaaattaggatcacatgggaagaggaacagtcatataaagaaatgcacagggctggagcgaattgcacgggattcatcttacgaacaagaaggaaaggttcaatttgtaattgatgcagtgtattccatggcttatgcactgcacaacatgcacaaagaactctgccctggttacataggcctttgcccaaggatggttaccatcgatgggaaagagctactgggttacatcagggccgtgaattttaatggcagcgctggtacacctgtcacttttaatgagaatggagatgctccgggacgctacgatatcttccaatatcagataaacaacaaaagtacagaatacaaaatcatcggccactggaccaatcaacttcacctaaaagtggaagacatgcagtgggctaatagagagcacacgcacccagcatctgtctgcagcctgccgtgcaagcctggggagaggaagaaaaccgtgaaaggggtcccttgctgctggcactgtgaacgctgcgagggttataactaccaggtggacgaactctcctgtgaactctgccctttggatcagagaccaaacatcaaccgcactggctgccagaggattcccatcatcaagttggagtggcattcaccctgggccgtggtacctgtgttcatagcaatattgggaatcattgccaccacctttgtgattgtgacctttgtccgctataatgacacaccaatcgtgagagcttctgggcgggaacttagttatgtgctcctaacggggatttttctctgttactcaatcacttttttgatgattgcggcacctgacacaatcatctgctctttccgaaggatcttcctgggacttggtatgtgtttcagctatgcagcacttttgaccaaaacaaaccgtatccaccgaatattcgagcaagggaagaaatctgtcacagcacctaagttcatcagcccagcatcccagctggtgatcaccttcagcctcatctccgtacagctccttggagtgtttgtgtggtttgtcgtggatcccccccacaccatcattgactatggagaacagcgaacactggatcccgagaacgccaggggagtgctcaagtgtgacatttccgatctgtcactcatttgttcactgggatacagtatcctcctgatggtcacttgtactgtttatgccattaaaaccagaggggttccagaaacgttcaatgaagccaaacctattggatttaccatgtacaccacgtgcatcatttggttagctttcattcccatcttttttggtacagcccagtcagcagaaaagatgtacatccagacaacaacacttactgtctccatgagtttaagtgcttcagtgtctctgggaatgctctatatgcccaaagtttatattataatttttcatccagagcagaacgttcaaaaacgcaagagaagcttcaaggctgtggtcacggccgctaccatgcaaagcaaactgatccaaaagggaaatgacagaccaaacggcgaggtgaaaagtgaactctgtgagagtcttgaaaccaacagtaagtcatctgtagactttcagatggtcaagagcgggagcacttcctaatagatcttcttctaccaagacaacatacatcagctacagtaatcattcaatctgaacagggagatggcaccatctgaaggaaggtgctgtatattcttaaatactgaacatgagactgccaaaacgtactctgggagatctccatagactacaatcaatcagatcaatagtctcgtaaggaacaaaaattagccatgagccaaaagtatcaatattgaggagcaaagaagcccatttttatacaatacaaccaatgagtgtcaagctaaagtattgcttacttgtgagcagttaaaaaaaaaatctcaaacggaaaactaatgttagcttgtggaaaaaaaaatgctgttgaaataaaccatgtctgatgttattcttgtaagtatttttctgtgattgtgagaactcctgttcctgtcccacactgttcaacttgtataagcctctgagtctgtttcttgtaatggctgactggattgaagccctgggttgtgctacaaaataaatgcaatgattgacgcatgcaatttttatacaaataatttatttctaataataaaggaatgttttgcaaatgttaaaaaaaaaaaaaaa

SEQ ID NO: 13 - DNA, 2793 bp, Rattus norvegicus
gctaccccttcttctgtggcaagaataaacttctggtcgctgactgcaataccccccgtggagaaaatggtatgcgagggaaagcgcttagcctcttgcccttgcttcttcctcttgactgccaagttctactggatcctcacaatgatgcaaagaactcacagccaagagtatgcccattccatccgggtggatggggacatcattttggggggtctttttcctgttcatgccaagggagaaagaggggtgccttgtggggagctgaagaaggaaaaggggatccacagacttgaggccatgctttatgcaatcgaccagattaacaaagaccctgatcttctctccaatatcactctgggggtccggatccttgacacgtgttccagggacacctacgctttggagcagtcactgaccttcgtgcaggcactgatagagaaagatgcgtctgatgtgaagtgtgctaatggagatccgcccatattcaccaagcccgacaagatttctggcgtcataggtgctgcagcaagctctgtgtccatcatggtggctaacattttaagactatttaagatacctcagattagctatgcatccacagccccagagctaagtgacaacaccaggtatgatttcttttctcgggtggtcccgcctgactcctaccaagcccaagccatggtggacattgtgacggccctgggatggaattatgtgtcaacactggcttccgaggggaactatggagagagtggtgttgaggccttcactcagatctcaagggagattggtggtgtttgcattgctcaatcacagaaaatcccacgtgaaccaagacctggggaatttgaaaaaattatcaaacgcctgctggagacacctaacgctcgcgcagtgattatgtttgccaacgaggatgacatcaggaggatattggaagcagcaaaaaaattaaaccagagtgggcattttctatggattggctctgatagttggggatccaaaattgcgcccgtctaccagcaggaggagatcgcagaaggagcggtgacaattttgcctaaaagagcatcaattgatgggtttgatcgatactttagaagccgaactcttgctaataatcgaagaaatgtgtggtttgcagaattttgggaggagaattttggatgcaaattaggatcacatgggaagaggaacagtcacataaagaaatgcacagggctggagcgaattgcacgggattcatcttatgaacaagaaggaaaggttcaatttgttattgatgcagtgtattccatggcttatgcactgcataacatgcacaaagagcgctgtcctggatacataggtctttgcccaagaatggtcaccatcgatgggaaagagctactgggttacatcagggccgtgaattttaatggcagtgctggtacacctgtcacttttaatgaaaatggagatgctcctggacgctacgatatcttccagtatcagataaacaacaaaagtacagagtacaaaatcatcgggcactggacaaatcaacttcatctaaaggtggaagatatgcagtgggctaatagagagcacacgcacccagcatctgtctgcagcctgccgtgcaagcccggggagaggaagaaaaccgtgaaaggggtcccttgctgctggcactgtgaacgttgtgagggttataactaccaggtggatgaactgtcctgtgaactttgccctttggatcagagaccaaacatcaaccgcacaggctgccagaggatccccatcatcaagttggagtggcattcgccctgggctgtggtacctgtgttcatagcaatattgggaatcattgctaccacctttgtgattgtgacctttgtccgctataatgacacgcccatcgtgagagcttctgggcgggaacttagttacgtgctcctaacggggatttttctctgttactccatcacctttttgatgattgccgcacctgacacgatcatctgctccttccgacggatcttcttgggacttggtatgtgtttcagctatgcagcacttttgaccaaaacaaaccgtatccaccgaatattcgagcaagggaagaaatctgtcacagcacctaagtttatcagcccagcatcccaactggtgatcaccttcagcctcatctctgtccagctcctcggagtgtttgtgtggtttgtcgtggatcccccccacaccatcattgactatggcgaacagcgaacgctggatcctgagaacgccaggggagtgctcaagtgtgatatttcggatctgtcactcatttgttccctgggatacagtatcctcctgatggtcacctgtactgtttatgccattaaaaccagaggggtcccagagacgttcaatgaagccaaacctattggctttacaatgtacaccacgtgcatcatttggttagctttcatccccatcttttttggtacagcccagtcagcagaaaagatgtacatccagacaacgacacttactgtttccatgagtttaagtgcttcagtgtctctgggaatgctctatatgcccaaagtttatattataatttttcatccagagcagaatgttcaaaaacgcaagagaagcttcaaggctgtggtcacagctgctaccatgcaaagcaaactgatccaaaagggaaatgacagaccaaatggcgaggtgaaaagtgaactctgtgagagtcttgaaaccaacacgtcttctaccaagacaacatacatcagctacagtaatcattcgatctga

SEQ ID NO: 14 - DNA, 4827 bp, Homo sapiens
caatcaggatgctggggctcggagatgatgggcggccgcctcctcactggagggattcctggagaagctgcagtgtggggatgctctagaccgagtactgcccccagtgtctttccacctcctcctgcagcagcggcagcggcagcagcagttagcagcggcaacttgaggctgcaccccgggcaagtccccagggtggtgctcagccgagaggggggcttggcgccccgaaggggtgtgtgtagggtgggggcgaccagctgggaccagctggtggccctggaaaacctcccacacacccacacccacacaccccttttgtgttgcaggctgcccctccaagagcggaggcagcgagagtacgcgtgtgcctcgcgccggtccacgcggggagagcactggggaccgagacccggcaccacctcccggtccgccctccagggaaaacgggaaaactagcaagagctagcaagaactagcaagagcttgaacaaacgcctggactcagattggaagactgctcatttgtctactgcctcattcctggaaattgcactggaactgtctgattaagaaaaacagaataattctgaaagaaagaaaacaaagaaaaacatactccagaattcctaatagaacacttcacctgaacctaaaatggtgagcgagagtcaccatgaggccctggcagccccgcctgtcaccactgtcgcgactgttctgccaagcaacgccacagagccagccagtcctggagaaggaaaggaagatgcattttctaagctgaaggagaagtttatgaatgagttgcataaaattccattgccaccgtgggccttaattgcaatagccatagtcgcagtccttttagtcctgacctgctgcttttgtatctgtaagaaatgtttgttcaaaaagaaaaacaagaagaagggaaaggaaaaaggagggaagaatgccattaacatgaaagatgtaaaagacttagggaagacgatgaaagatcaggccctcaaggatgatgatgctgaaactggattgacagatggagaagaaaaagaagaacccaaagaagaggagaaactgggaaaacttcagtattcactggattatgatttccaaaataaccagctgctggtagggatcattcaggctgccgaactgcccgccttggacatggggggcacatctgatccttacgtgaaagtgtttctgctacctgataagaagaagaaatttgagacaaaagtccaccgaaaaacccttaatcctgtcttcaatgagcaatttactttcaaggtaccatactcggaattgggtggcaaaaccctagtgatggctgtatatgattttgatcgtttctctaagcatgacatcattggagaatttaaagtccctatgaacacagtggattttggccatgtaactgaggaatggcgtgacctgcaaagtgctgagaaggaagagcaagagaaattgggtgatatctgcttctcccttcgctacgtacctactgctggtaagctgactgttgtcattctggaggcaaagaacctgaagaagatggatgtgggtggcttatccgatccttatgtgaagattcatctgatgcagaatggtaagaggctgaagaagaaaaagacaacaattaaaaagaacacacttaacccctactacaatgagtcattcagctttgaagtaccttttgaacaaatccagaaagtgcaggtggtggtaactgttttggactatgacaagattggcaagaacgatgccatcggcaaagtctttgtgggctacaacagcaccggcgcggagctgcgacactggtcagacatgctggccaaccccaggcgacctattgcccagtggcacaccctgcaggtagaggaggaagttgatgccatgctggccgtcaagaagtaaaggaaagaagaagcctttctgcatttgcccatatagtgctctttagccagtatctgtaaatacctcagtaatatgggtcctttcatttttccagccatgcattcctaacacaattcagtggtacttggaatcctgttttaatttgcacaaatttaaatgtagagagcccctaagtccttcatcataccactgccctccaaatctactcttcttttaagcaatatgatgtgtagatagagcatgaatgaaattatttattgtatcacactgttgtatataccagtatgctaaagatttatttctagtttgtgtatttgtatgttgtaagcgtttcctaatctgtgtatatctagatgtttttaataagatgttctattttaaactatgtaaattgactgagatataggagagctgataatatattatacggtaaatatagtatcgtctgcattccagcaaaaatatcaactcgtaaggcactagtacagttaaactgacatcttaaaggacaacttaaacctgagctttctattgaatcatttgagtaccaagataaacttacaccacatacttggtgggtgaatccaattttgtagaattcctacacaggcaaaatagcatgatctgagcagcagcatccaggctgacctcaaggaagcatagccacaaaacagaatagcacctgtctgtacatatttacaaagctaaaataatggcttcactcttatatttgaggaagcaactgaacaggagtcaatgatttcatattactgcatatagaataacaacaaggtgttccgtgtgtgtgtgtgtgtgtgtgtgtgtgtgtgtgtgcacatttgtttggggatgggggagaagaagctaaggggagaagtcaacatttatgaaatattgcctgactatttaaaaagaaaaaagtagctctccattatcacctttatacaaaatgtacatcctgtgaattctgttccagatttcacacctacaataattccaaaaggtttgcacattagagtttgtaacaaaatattttattatataaaaccaggttagaaggaatgcaggatatttttaacacaacaatctgtgcttattacacaaaattactttgtggtaaacagacagtattgtaatcccatcaaaagatgaaagaaaaacaaaaacaaaaaccaacaacaattagccatagttctgaatgcacttcaattaagccaaaacagacagctagtgatctttttatatgctctttttacttaagttttaatttgtcctttaaaaaaaggtgaaacaaaccaagaacaagttctagaaaactgaagcaacctcttatgtatactagatgcttgatttaggaggagtttttaaacgttttcaatgttattatgtagtaaatgacactattatgaagctactagtcattccataagagtcttaaaggactgctctgtgtaacactgtgactgccgtgtgtgcttagacccgtagtttcctcagtggatagcactcaatttattccgtagtgatattgtaacaatactgccattcccttctactgcactgcccaaggtgtgtgtagcacaaacagttctcattacaaaggaccaattcagaactgaaaagctatgcataggacaaggaagatacatagaatggggtggaacacagcattttgtcaagcactgtgcaatattccatatttttccccactatggtagacaaccatttcgtggaagggcagcctattatcccacactgcatctagccttttgtcccattcacttctgtgatccattttaatttccaggccacaagacagtagtgatgctctgaaatgaaagtttgtcttcacaaatatcaaaacaaaatggaggaaaactaagcattggcctcatgttcagtcttcaggatatcacaccacgtcttttcaaaaactaaagagaattcaaaaagggctgatggtaggctttgaacatggggttggctgtttcccagtaaaactggaattcctgtcgttactgtttccttatcaaagaaggggcaagctcttttgccttttaggccagacatagcaaacgctttataattggcatagacataaaggataaaaggaaaataaccgtctgccgatggtccgtacttcttaaaaaacataggtaatagaaaatatacacaagtcagaatgtgaaattaaataatggtttgaacagaaaattcaaacaagactctttccaatttaaagggccaaaccctaccaaagagagggagttgactggcttttaaaaagtatttaaataccacaaatgacatttaatttcactgtattcagctttaagttgttcacaatgaaaccacactttcaaacaagcaggttcaagctgctgaatagacattatttcttgcattaaaataccactaatgcattctcttgcaacactgccagacatgggattgtcaccatagaattagttggtactatgccatctttcactctttcacaagtcagtgatggaacctgctttatgaccaagattcatcctcaaataagccacatgtacccttctgacaaagctgtgtaaagtattagaatctgatgctctagaaagatcctagttgcctttgtgtatatttactgcctgcttgagtgtttctatgtgtgggttttccctgtatcttgtagaaatgttggggtgttttcctctgccatatggctcgtggcctgcgagccaactatttcagctgtattttaccttcatttttgatgaggtgatttaaattttgtttcactttgtgtagtgaattccacagtagttttctgattgttgttaaaaatgacttaacatattacacagatattcaataaaaatgttttatttcctgttgaaaaaaaaaaaaaaaaaa

SEQ ID NO: 15 - DNA, 4836 bp, Mus musculus
tcctcactggagggattcctggagaagctgcagtgtggggatgctctgaccgagttcagcccccagtgtctttcctcctcctcctcctgcagcgacagcatccgcagttagcagtgggcaacttgaggctgtaaccggggcaagcccccagggtcctgctcacccaacagggggctcaggtccccgaaggggtgtgtgcagggcgggggcggccagctgggaccagctggtggccctagaaaacctcacccacacccacacccacacaccccttttgtgttgcaggctgcccctctgagagcggaggcagcgagagagtactcgtgtgcctcgcaccggtccgcggtgagagcagcggggaccaagactcgcaccatctcccggtcggtcctcgctccagtttccctctgaatcctacacttcatatgtagacaccttactcaactggcatttgttagtcaagtctcctctgcatccaaggaaaagaagactttggcgcgctcgaacaaccaacatccgcagtctgatcagaagacattgcccttgccgtcccgagagctccagcagaacatctcgttaagattgaagaaaggagattccaaaaggaacaaaaaaacccaaatactccagactaccccagcagaacattccgcttgaaccaaaaatggtgagtgccagtcgtcctgaggccctggctgcccctgtcaccactgttgcgacccttgtcccacacaacgccactgagccagccagtcctggggaagggaaggaagatgccttttccaagctgaagcagaagtttatgaatgaactgcataaaatcccattgccaccgtgggccttaattgccatagccatagttgcggtccttctagtcgtgacctgctgcttctgtgtctgtaagaaatgtttgttcaaaaagaaaaacaagaagaagggaaaggaaaagggagggaagaacgccattaacatgaaagacgtgaaagacttagggaagaccatgaaggatcaggcccttaaggatgacgatgctgaaactggactgactgatggagaagaaaaggaggagcccaaggaagaggagaaactgggaaagcttcaatattcactggactatgacttccagaataaccagctgctggtgggaatcatccaggctgctgaactgcccgccctggacatgggaggcacatctgatccatacgtcaaagtcttcctgctgcccgacaaaaagaagaagtttgagacaaaagtccaccggaaaaccctcaatccagtcttcaatgaacagtttactttcaaggtgccatactcggaattaggtggcaagacactggtgatggctgtgtatgattttgaccgcttctccaagcacgacatcattggagagttcaaagttcctatgaacaccgtggattttggccacgtcaccgaggagtggcgcgatctccagagtgctgagaaagaagagcaagagaaactgggtgacatctgcttctccctccgctacgtccctactgccggcaagctgactgttgtcattctggaagccaagaacctgaagaagatggatgtgggtggcttatctgatccctatgtaaagattcacctgatgcagaacggcaagagactgaagaagaaaaagacaacgattaagaagaacacacttaacccctactacaatgagtccttcagctttgaagttccgttcgagcaaatccagaaagtgcaagtggtggtaactgttttggactatgacaagattggcaagaacgacgccatcggcaaagtctttgtgggctacaacagcaccggcgcagagctgcgacactggtcagacatgctggccaacccccggcgacccatcgcccagtggcacactctgcaggtagaggaggaggttgatgccatgctggctgtcaagaagtaaaggggaaaagaagcctttctgcgtctgcccacatagtgctctttagccagtatctgtaaatacctcagtaatatgggtcctttcagtttccagccatgcattcctgatacaatccagtggtacttcagatcctgttttaatttgcacaaatttaagtgtagaaagcccctatgcccttcatcataccactgccctccaaatctactcttcttttaagcaatatgatgtgtagatagagcatgactgaaatgtattgtatcacaccgttgtatataccagtatgctaaagatttatttctagtttgtgtatttgtatgttgtaagcgtttcctaatctgtgtatatctagatgtttttaataagatgttctattttaaactatgtaaattgactgagatataggagaactgataatatattatatggtaaatatagtatcgtctgcattccagcaaaaatatcaatttgaaaggcactagtacagttaaaccaacatcttaaaggacaacttaaacctgaactttctattgaatcctttgagtaccaagatttgctcacatgacatctttgatgggtgaacccaattttgtagaattctttcacaggcaaatagcatgttctgagcagcatccatgctgacctcaaggaagcaaagccacaaaccagaatagcatctgtctgtctgtctctgcaaagctaaaaaatcatggcttcactcttccagtcaaggaagcaacgaacacgaaggagccagtgagttcctacgactgcatctagcatagcataaccgtggtgttctctgtgtgtgcgtgtgcaagtgtgaaagtgtatgcacgagtcttatgtgtggtgcatgcctttgttcggggttggggtgggagaggagaagctgagagaagtcagcgtttctgaaatgttgcctgactacttaaacagaaactaatagctctccagtatcacatttatataacacgtgcaacctgggaattctggtccagatttcaccccaatattgattccaaaaggtatgcacgtgagactttgtaacaaaatattttattatataaaaccagattagaaggaatgcagaatatttttaacacagcaatctgtgcttattccacaacattactttgtggtaaacagacagtattgtaatcccatgaaaagatgaaatataacaattagccatagttctgaatgcacttcgattaagccaaaacagacagctagtgatctttttatatgctctttttacttaagttttaatttgtcctttaaacaaaggtgaaaaaaaaaaccaagaacaagttctagcaaactgaagcaacctcttatgtacactagatgcttgacttaggaggagtttttaaatgttctcaatgttattctgtagtaaatggcactattatgaagccactagtcattccatatgagtcttaaggactgctctgtgtaacactgtgactgccctgtgtgcttagacacgtagtttcctcagtggctagcacccaacttactccgtagtgatattgtaacaatactgccattccctcgtactgcactgcccaatgtgtgtgtagcacaaacagttctcattcttaaggaccaattcagaactgaacagctatgcataggacagaaagatacatagactgggggggtggggagaacacacagcatcttgtcaacactgtgcactagtccacatttgtctcgctgaggtagacagccacttcaggaaggtgagcctgctacctaacactgcttctagactcttcgcccacttgatagtgcggcctgttttcacctccaggtcacagaaaacagcaacatcctgacgggagaggaccatcttcacatctaccaaaataaaatggaggcatggaaagtgtggcctcctgtttgatctataggatttagctccatgctttggacaaaagtcaagagaatcccagaaagggctcttggtagactttaaacatggggttggccgttcctcctgcacacacaacacaatctccaaaccctggttcttccctcattggagaagaggcaaagcctttgctcctcacctccatcccagcaaactcttcagaattagaacagatgcagaggacaaggagaaaatcctttcctgtggacgaacagcacattttcaacattaaaaccacagatgataggaaaggcacactcataggtgagattaaacaggagactcaaatgaggggctttcctacttaagggaccaagtcctaccaaagagaaggagttgactttaaaaaaaaaaataccttagataccagacaccgacatttcggttcaccacctcccgacttaagttgtacacggtggagcaacatttccaaggaagcagcttccagctgcccaagcagacattccttcctgcatcacaacactactagcacattctcttgcaacactgccaacgtgggattgtcaccatagacttagttggtactatatcatctcccatgagccggtgactggacctgcttcctgacctgccatccatcctcagatgagccacctgtacccttctgacagtgcagtgtgaagtcttagaagctgatgccctagaaagatcctagttgcctttgttgcatatatttactgcctgcttgagtgtttctctgtgtgggttctctctgtgtctcgtagaaatgttggggtgttttcttctgccataagggctcgtggcccgcgagccaactcctttagctgtattcttccttcattttttgataagtggtttaaattctgtttcactttgtgtagtgaaccccatggtagttttctgatcgttgttaaaaaaaatgacttaacatattacatggacactcaataaaaatgttttatttcctgttgaaaaaaaaaaaaaaaaa

SEQ ID NO: 16 - DNA, 1896 bp, Rattus norvegicus
ctggtggccctagaaaacctcacccacacccacacaccccttttgtgttgcaggctgcccctctgagagcggaggcagcgagagtactcgcgtgcctcgcaccggtccgcggtgagagctgcggggaccaagactcgcaccacctcccggtcctcgctccaggaaaagaagacttgaaagcgcttgagcaaccaacatccgcagtcagatcggaagactttgccctggccatccccagagcgccaccagaacgtctcattaagattgaagaaagattccgagaagaacaaaaccccccaaatactccataataccctgcagaacatttcacttgaaccaaaaatggtgagtgccagtcatcctgaggccctggccgcccctgtcaccactgttgcgacccttgtcccacacaatgccactgagccagccagtcctggggaagggaaggaagatgccttttccaagctgaagcagaagtttatgaatgagctgcataaaattccattgccaccgtgggccttaatagccatagccatagttgcggtccttttagtcgtaacctgctgcttttgtgtctgtaagaaatgtttgttcaaaaagaaaaacaagaagaaggggaaggaaaagggaggaaagaacgccattaacatgaaagacgtgaaagacttagggaagaccatgaaggatcaggcccttaaggatgacgatgctgaaaccggactgactgatggagaagaaaaggaagagcccaaggaagaggagaaactgggaaagctccaatattcactggactatgacttccagaataaccagctgttggtgggaatcatccaggctgctgaactgcccgccctggacatggggggtacatccgatccatacgtcaaagtcttcctgctgcctgacaaaaagaagaaatttgagactaaagtccaccggaaaaccctcaatccagtcttcaatgaacaatttactttcaaggtgccctactcggaattaggtggcaaaaccctggtgatggctgtgtatgactttgatcgcttctccaagcatgacatcatcggagagttcaaagttcctatgaacaccgtggattttggccatgtgaccgaggagtggcgtgatctccagagcgctgagaaagaagagcaagagaaactgggtgacatctgcttctccctccgctacgtccctactgccggcaaactgactgttgtcattctggaagccaagaacctgaagaagatggatgtgggtggcttatctgatccctacgtgaagattcacctgatgcagaacggtaagaggctgaagaagaaaaagacgacgattaagaagaacacactcaacccctactacaacgagtccttcagctttgaagttccgttcgagcaaatccagaaagtgcaagtggtggtaactgttttggactatgacaagattggcaagaacgacgccatcggcaaagtcttcgttggttacaacagcactggggcggagctgcgacactggtcagacatgctggccaacccccggcgacccatcgcacagtggcacactctgcaggtagaggaggaggttgatgccatgctggctgtcaagaagtaaagggaaaacgaagcctttctgcatctgcccacatagtgctctttagccagtatctgtaaatacctcagtaatatgggtccttttggtttccagccatgcattcctgatacaatccagtggtacttcaaatcctgttttaatttgcacaaatttaagtgtagaaagcccttatgccctccatcataccactgccctccaaatctactcttcttttaagcaatatgatgtgtagatagagcatgactgaaattatgtattgtatcacactgttgtatataccagtatgct

SEQ ID NO: 17 - DNA, 4070 bp, Homo sapiens
gtccagcctgtttaaacggaaaggacagagatcctgtctgttcaatgtaaaaaaaaaaaaaaaaaaaaaaaaaagaaaaaaagaaaaaaaaaatcagatcagaccgagagagagaggagagagggagagagagagggagagagagagggagagagagaggagagggagggagggagagagagaggggggagagcagagagagagcgcgagcgcgagcgagcgagagaagaggagaaagagagagagcagagagcgagcggagagcgaggtgtagagaaaccgagggggagagaacccgagtgtgtgtatgcgtgtgcgtgtgtgagcgcgagcgagcgagagagaggagcgagagagtgtgagcgagaaagaataaaaggaaagaagattttctctatgtatataaagatggccacgttagcaaacggacaggctgacaacgcaagcctcagtaccaacgggctcggcagcagcccgggcagtgccgggcacatgaacggattaagccacagcccggggaacccgtcgaccattcccatgaaggaccacgatgccatcaagctgttcattgggcagatcccccgcaacctggatgagaaggacctcaagcccctcttcgaggagtttggcaaaatctacgagcttacggttctgaaggacaggttcacaggcatgcacaaaggctgcgccttcctcacctactgcgagcgtgagtcagcgctgaaggcccagagcgcgctgcacgagcagaagactctgcccgggatgaaccggccgatccaggtgaagcctgcggacagcgagagccgaggaggtagtagctgcctgcgccagcccccttcacaagatagaaaactcttcgtgggcatgctcaacaagcaacagtccgaggacgacgtgcgccgccttttcgaggcctttgggaacatcgaggagtgcaccatcctgcgcgggcccgacggcaacagcaaggggtgcgcctttgtgaagtactcctcccacgccgaggcgcaggccgccatcaacgcgctacacggcagccagaccatgccgggagcctcgtccagtctggtggtcaagttcgccgacaccgacaaggagcgcacgatgcggcgaatgcagcagatggctggccagatgggcatgttcaaccccatggccatccctttcggggcctacggcgcctacgctcaggcactgatgcagcagcaagcggccctgatggcatcagtcgcgcagggcggctacctgaaccccatggctgccttcgctgccgcccagatgcagcagatggcggccctcaacatgaatggcctggcggccgcacctatgaccccaacctcaggtggcagcacccctccgggcatcactgcaccagccgtgcctagcatcccatcccccattggggtgaatggcttcaccggcctccccccacaggccaatgggcaacctgctgcggaagctgtgttcgccaatggcatccacccctacccagcacagagccccaccgccgcggaccccctgcagcaggcctacgccggagtgcagcagtatgcaggtccagctgcctaccctgctgcctatggtcagataagccaggcctttcctcagccgcctccaatgatcccccagcagcagagagaagggcccgagggctgtaacctgttcatctaccatctgccccaggagtttggggacgctgagctgatgcagatgttcctccctttcggcttcgtgagcttcgacaacccggccagcgcgcagaccgccatccaggccatgaacggcttccagatcggcatgaagaggctcaaggtgcagctgaagcggcccaaagacgccaatcgcccgtactgagcgccggcgggagcgtcccccgggggagaccaggactcgcacagggcaggatgctgaacgggctacattaaaaaacaaacctctctctatatatatttataaatgagaactgttggatgacacctttgacatatcagccaatatcaatcaagctgaagactccagacactgtctgtgtgactgtaacatttcttcaaggaaagtatagcgtctatggagttcagagggcacgtgtttgggggaaaatatatatgacatgaagaagaagatgaagaaaaatgagaaaaaaacacacaaaaggcaactttaaaacaaaatatcacgagcagacggggaggctgaagggctgggagctgggaggagacgctgcttaccgatcccggggcttttccagcccacgggcgcctgacgcaggctggggcaagtggtgcgtggggcctggtccccaaggggcggctgagaggccgccactgagcatctctatctgtcattcctttagctatttagggaccaaaggaccaaactttttattgcagatgtgtagctctatgtcaaatagagggggaatggaggaccccctccttcctgcctcatggctgttcttgaaacagcttagagcgattctatgaaaaaatgtaataaaaaattaaaaaaaaaacaaaaaacaaaaaaaacaacaaaaaaaggaaaaataacgcttcaatgcttttaaaacagcaagataatagttctttgatactttgagaggcgctttgatgaccctcatccaagtctatgacactttcctatggttttctgtattctatgtctggatggagctgttaaaagatgaacaaattggtggatatttggggaaagcaacacaaatcttaaaactcacccgtgaagtgtgagaaaacaaggaggggaacaaatgggacttaccaagcaaggtcattgttgtgaaaagtctgtaaatgcttctaactcttccccctcttaaaatcataatagttgtacagaattttaaaaaggaaaagtttaaaatacctatataatagaagaaaaattagaggaaagcaaaaaataaaaaaaaaaaataaaaaaggaaaaaaaaaacctatagaagttagcattactgctaaaatcccagatgttgtaggactgtacttttgtagagtttagacagagcatcaatcccttctccccgcgagtgcagtcggatgccgttgtccccaagggccaggccgaactcgccccacacctgcgggcgcctggctgccgacgccggtggaggccacgccgtcggtcgggaacggtcgcgtcgcccgggtcgccgccgccgccgccgccgtcatcctcgcctctcgcttccctctgggagctgcgcgcgcttcactcaccttccgcttcactcgccttccacgcttgctccagagactttccgggcaagggagaactgggaactttcatcttaaaacaactagacaacacacacacacacaaaaccttaaaaaagagagagaaaaataaacaatctttgaagaatttctgaaactgtttacaaggaattttgaaaaacagggatgtttaaatgatgccggctctgtttttctgtaaataaatttgcatattttgtaggacttttaattgtaatgatagagaaaaaaacaaggaaaactatttaatgaaagcacgatatttatctttggtaaatgactttagagcagttaaagacattgcttaaaaaactcagaatcagaaaaaacactgaattatttaagaacacatatattttaaagtataacatatttattataatttatttgcacccttgggaagattgctttggcattctgccttgattccttctcattgatgtccaggtcatctggaggccgggggttctcagacctaccagtttccctttagaagaacaaatttccagttcctctagggcctcctgcctttcctttctttctgtcaattttgttgttgttgttgtttttcattttctttttttctcttgactcctccttttaggatgtccagatgtaaaaaaaaaaaaaaaaaaaagaaaaaaagaaaaaaaaaagaaaacagctgcagttcagtacaactgctcttttcacactcaactccctaaaactccttgtaaccttctgtaactattggatgacgctttctccagcttagccctaaataaagcacagtttaaaaaaaaaaaaaaaaaa

SEQ ID NO: 18 - DNA, 4007 bp, Mus musculus
aagaagaagagggagagagagagagagagagagaagagagaagagagagagaagagagagagaagagagaaatagaacgagagcccagagcagagcaaggtgtaggaaaccaaaggggagagaacccgagtgtgtgcgtgtgtgtatgagtgtgtgtgagtgtgtgtgtgtgtgtgcgcgcgtgtgtgagtgtgtgtgtgtgagtgtgtgtgtgtgtgtgtgtgagagtgtgtgtgcgtgtgtgtgtgtgtgtgagtgtgtgtgtgtgcgtgtgcgtgtgtgagcgcgagcgagcgagtgagggagaggagcgagagagtgcgagcgagaaagaataaaaggaaagatttttctctatgtatataaagatggccacgttagcaaacggacaggctgacaacgcgagcctcagtaccaacgggctaggcagcagcccgggcagcgccgggcatatgaacggattaagccacagcccggggaacccgtcgaccattcccatgaaggaccacgatgccatcaagctgttcattgggcagatcccccgaaacctggatgagaaggacctcaagcccctcttcgaggagttcggcaagatctacgagcttacggttctgaaggacaggttcacaggcatgcacaaaggctgcgctttcctcacctactgcgagcgtgagtcagcgctgaaggcccagagcgcgctgcacgagcagaagaccctgcccgggatgaaccggccgatccaggtgaagcctgcggacagcgagagccgaggaggtagtagctgcctgcgccagcccccttcacatagaaaactcttcgtgggtatgctcaacaagcaacaatctgaggacgacgtgcgccgcctcttcgaggccttcgggaacatcgaggagtgcactatcctgcgcgggccggacggcaacagcaaggggtgcgcctttgtgaagtactcctcccatgccgaggcacaagccgccatcaacgctctacatggcagccagaccatgcctggagcctcctccagcctggtggtcaagtttgcagacactgacaaggagcgcacaatgcgacggatgcagcagatggctggccagatgggcatgttcaaccccatggccatccccttcggagcctatggcgcctatgctcaggcactgatgcagcagcaagcagccctcatggcatcggtcgcgcaaggaggctacctgaatcccatggctgccttcgctgccgcccaaatgcagcagatggcggccctcaacatgaatggcctggcagccgcacctatgaccccaacctcaggtggcagcacccctccaggcatcactgcaccagctgtgcctagcatcccatcccccattggggtgaacggcttcacgggcctcccccctcaggccaatgggcagcctgctgcggaagctgtgtttgccaatggcattcacccctacccagcacagagccccaccgcagccgaccccctgcagcaggcctacgctggagtgcagcagtatgcaggacctgcctaccctgctgcctatggtcagattagccaggcctttcctcagccaccgccaatgattccccagcaacagagagaagggcccgagggctgtaacctgctcatctaccatctgccccaggagtttggggacgctgagctgatgcagatgttcctccctttcggtaatgtcatctcctcgaaagtgtttgtggatcgggcgactaaccaaagtaaatgctttggcttcgtgagcttcgacaacccggccagcgcacagaccgccatccaggccatgaacggcttccagataggcatgaagaggctcaaggtgcagctgaagcggcccaaagacgccaatcgcccgtactgagcgccggcgggagcgtcccccgggggagaccaggactcgcacagggcaggatgctgaacgggctacattaaaaacaaacctctttctctctctatttataaatgagaactgttggacgacacctttgacatatcagccaataccaatcaagctgacgactccagacactccgtgtgactgtaacatttcttcaaggaaagtatagcgtctatggagtgcagagggcatgtgtttggggaaaaatatacatgacataaagatgacgacgaagaagaaaaatgatataaaacaaacaaaaaacccgcgactttaaaatgaaataacgtgagcccagatggggaagctgaagggctgggcactaggaggagatgctgctgccaaccgatcctggggcttttcccgcccgtaggcgcctgaggcaggcaggggcaagtgtacaatggggcctagtccccagcaggcggctgggaggccgccactgagcatctctatttgtcattcctttagctatttagggaccaaaggaccaaactttttattgcagatgtgtagctctgtcaattagaggtggaatggagaccaacctccttcctgcctcctggctgttcttgaaacagcttagagcgattctatgaaaatgtaataaaaaataaaaataaaaaaaaaataaaaaaagaacaaaaaaaacaaggaaaaaaaataatgcttcaacgcttttaaaacagcaagatactagttctttgatactttgagaggcgctttgattaccccgattgaagtctctgaaactttcctatggttttctgtattatatgtctggatggagctgtttagatacattgattggtggatatttggagaaagcaacaaagatattaagtcattaaccatgaagagaacacgaggggaaccaggggacttcgaaggcaaggtaattgttgtgaaaagtctgtaaatgcttctaattcttcccccctcttaaaatcataatagttgtacagaattttaaaaggaaaagtttaaaatacctatatcatagaagaaaaaaaaattagaggaaagcaaaaaaataaaaaaaaaaaggaaaaaaaaaccttatagaagttagcattatgctaaaatcccagatgttgtaggactgtacttctgtagagtttagactgagcatcgatcccgtctccccgcgagtgctgtcggactccgtccccaagggccaggccggactgtccagacctgcaggctccggccagaagcgccgctccggaagcgtcctgccgcctctcgctttgccagcctgggagccgcgcgcatgcgcgctctctctgcccttctcacctctgcttcactcgccttctgtgcttgttccggagactttccaggccagggagaactgggaacttctcatcttcaaacaactagacaacacacacacacacaaaaccttaaacaagagagagaaaacaaacaatctttgaagaatttctgaaactgtttacaaggaattttgaaaaacagggatgtttaaatgatgctggctctgtttttctgtaaataaatttgcatattttgtaggacttttaattgtaatgatagagaaaaaacaaggaaaactatttaatgaaagcacgatatttatctttggtaaatgactttagagcagttaaagacattgcttaaaaactcagaatcagaaaaacactgaattatttaagaacacatatattttaaagtataacatatttattataatttatttgcaccttgggaagacttgctttgcattctgcctcaagagcttctcattgatgtccatctggaggccaggggggtcttggaactaccatttttttttcctttagatcaacaaaattctggttgccctcaggcctccttccttttctttccatctattttgttgttgttttacttcatttagttttctttttctttctcttgactcctccttttaggatgtccagatgttgtaaaaaaacaaaaacaaaaaacccaacagctgcagttcaatacaactgctcttttcacacgactccctaaactccttgtaaccttctgtaactattggatgacgctttctccagcttagccctaaataaagcacagtttaaaaaaaaaacc

SEQ ID NO: 19 - DNA, 3473 bp, Rattus norvegicus
agaaatagaacgagagcccagagcagatcaaggtgtaggaaaccaaaggggagagaacccgagtgtgtgcgtgtgtgtgtgagtgtgtgtgagtgtgtgtgtgtgtgtgtgcgtgtgtgagtgtgtgagtgtgtgagtgtgtgagagagtgtgtgtgtgcgtgtgtgtgtgtatgtgtgtgtgtgtgcgtgtgcgtgtgtgagcgcgagcgagcgagtgagggagaggagcgagagagtgcgagcgagaaagaataaaaggaaagatttttctctatgtatataaagatggccacgttagcaaacggacaggctgacaacgcgagcctcagtaccaacgggctaggcagcagcccgggcagcgccgggcatatgaacggattaagccacagcccggggaacccgtcgaccattcctatgaaggaccacgatgccatcaagctgttcattgggcagatcccccgaaacctggatgagaaggacctcaagcccctcttcgaagagttcggcaagatctacgagcttacggttctgaaggacaggttcacaggcatgcacaaaggctgcgctttcctcacctactgcgagcgtgagtcagcgctgaaggcccagagcgcgctgcacgagcagaagaccctgcccgggatagaaaactcttcgtgggtatgctcaacaagcaacaatctgaggacgacgtgcgccgcctcttcgaggccttcgggaacatcgaggagtgcactatcctgcgcgggccggacggcaacagcaaggggtgcgcctttgtgaagtactcctcccatgccgaggcacaagccgccatcaacgctctacatggcagccagaccatgcctggagcctcctccagcctggtggtcaagtttgcagacactgacaaggagcgcacaatgcgacggatgcagcagatggctggccagatgggcatgttcaaccccatggccatccccttcggagcctatggagcctacgctcaggcactgatgcagcagcaagcagccctcatggcatcggtcgctcaaggaggctacctgaatcccatggctgccttcgctgccgcccagatgcagcagatggcggccctcaacatgaatggcctagcagccgcacctatgaccccaacttcaggtggcagcacccctccaggcatcactgcaccagctgtgcctagcatcccgtcccccattggggtgaacggcttcaccggcctccccccacaggccaatgggcagcctgctgcggaagctgtgtttgccaatggcattcacccttacccagcacagagccccaccgcagcggaccccctgcagcaggcctacgccggagtgcagcagtatgcaggacctgctgcctaccctgctgcctatggtcagataagccaggcctttcctcagccaccgccaatgattccccagcaacagagagaagggcccgagggctgtaacctgctcatctaccatctgccccaggagtttggggacgctgagctgatgcagatgttcctccctttcggcttcgtgagcttcgacaacccggccagcgcccagaccgccatccaggccatgaacggcttccagataggcatgaagaggctcaaggtgcagctgaagcggcccaaagacgccaatcgcccgtactgagcgccggcgggagcgtcccccgggggagaccaggactcgcacagggcaggatgctgaacgggctacattaaaaacaaacctctttctctctctatttataaatgagaactgttggacgacacctttgacatatcagccaataccaatcaagctgaagactccagacactccgtgtgactgttaacacttcttcaaggaaagtatagcgtctatggagtgcagagggcacgtgtttggggaaaaatatacatgacataaagatgccgacgacgaagaagaaaaatgatgtaaagcaagcaagcaaacaaacaaacaaacaaaaaccctcgactttaaaacgaaataacgtgagcccagttggggaagctgaggggctgggcactaggaggagatgctgctgccaaccgatcctggggcttttccagcccgtaggcgcctgaggcaggcaggggcaagtgtacaatggggcctcgtccccagcaggcggctgggaggccgccactgagcatctctatttgtcattcctttagctatttagggaccaaaggaccaaactttttatcgcagatgtgtagctctgtcaattagaggtggaatggagaccaacctccttcctgcctcctggctgttcttgaaacagcttagagcgattctatgaaaatgtaataaaaaaaataaataaaaaaaataaaaaagaacaaaaaaaaaacaggaaaaaaaataatgcttcaacgcttttaaaacagcaagatactagttctttgatactttgagaggcgctttgattaccccgattgaagtctctgaaactttcctatggttttctgtattatatgtctggatggagctgtttagataaattgattgatggatatttggggaaagcaacaaagatattaagtcagtaaccataaagagaacaggaggggaaccaggggacttcgaaggcaaggtaattgttgtgaaaagtctgtaaatgcttctaattcttccccctcttaaaatcataatagttgtacagaattttaaaaggaaaagtttaaaatacctatatcatagaagaaaaaaaattagaggaaagcaaaaaataaaaaaaaaggaaaaaaaaaccttatagaagttagcgttatgctaaaatcccagatgttgtaggactgtacttctgtagagtttagactgagcatcgatcccgtctccctgcgagtgctgtcggactcggtccccaatggccaggccggactgtccagacatgcaggctccggccggaagcgccgctccggaagcgtcctgccgcctctcgcttcgccctcctcacctctgcttcactcgccttctgtgcttgttccggagactttccaggccagggagaactgggaacttttcatcttcaaacaactagacaacacacacacacacaaaaaccttaaacaagagagagaaaacaaacaatctttgaagaatttctgaaactgtttacaaggaattttgaaaaacagggatgtttaaatgatgccggctctgtttttctgtaaataaatttgcatattttgtaggacttttaattgtaatgatagagaaaaaacaaggaaaactatttaatgaaagcacgatatttatctttggtaaatgactttagagcagttaaagacattgcttaaaaaactcagaatcagaaaaacactgaattatttaagaacacatatattttaaagtataacatatttattataatttatttgcacccttgggaagacttgctttgcattctgcctcaagagctcattgatgtccatctggaggccaggggggtcttggaact

SEQ ID NO: 20 - DNA, 1641 bp, Homo sapiens
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactggggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaaaaaaaaaaaaaaaaaa

SEQ ID NO: 21 - DNA, 1227 bp, Mus musculus
gtcattgagtgagcccggcagcgtccgtggtagcaggccatggcgatgggcagtggcggagcgggctccgagcaggaagatacggtgctgttccggcgtggcaccggccagagtgatgattctgacatttgggatgatacagcattgataaaagcttatgataaagctgtggcttcctttaagcatgctctaaagaacggtgacatttgtgaaactccagataagccaaaaggcacagccagaagaaaacctgccaagaagaataaaagccaaaagaagaatgccacaactcccttgaaacagtggaaagttggtgacaagtgttctgctgtttggtcagaagacggctgcatttacccagctactattacgtccattgactttaagagagaaacctgtgtcgtggtttatactggatatggaaacagagaggagcaaaacttatctgacctactttccccgacctgtgaagtagctaatagtacagaacagaacactcaggagaatgaaagtcaagtttccacagacgacagtgaacactcctccagatcgctcagaagtaaagcacacagcaagtccaaagctgctccgtggacctcatttcttcctccaccacccccaatgccagggtcaggattaggaccaggaaagccaggtctaaaattcaacggcccgccgccgccgcctccactaccccctccccccttcctgccgtgctggatgcccccgttcccttcaggaccaccaataatcccgccaccccctcccatctctcccgactgtctggatgacactgatgccctgggcagtatgctaatctcttggtacatgagtggctaccacactggctactatatgggtttcagacaaaataaaaaagaaggaaagtgctcacatacaaattaagaagttcagctctgtctcaggagatggggtgtcggtgtccctggtcgacaagaacagacgtctcctcgtcatcagtggactcttggctaagtggtgtcgtcatcagcatctccccgctgtgggagtccatccatcctaagtcagcagcagagcgtgcctggggcgtgagcagttggagggaccgaccagtggagtgtgcgtgtcggaaggcagtctacccagtcgtgactgagcacaaatgtgcaattgtcattttcttagcatgtcaagatttttattaatgcctttagaattaaataaaagtccttttttgaaatcttg

SEQ ID NO: 22 - DNA, 1243 bp, Rattus norvegicus
attttgggcgagcccagccccgtccgtggtagcaggccatggcgatgggcagcggcggcggcgcgggctctgagcaggaagacaccgtgctgttccggcgtggcaccggccagagtgatgattctgacatttgggatgatacagcattgataaaagcttacgataaagccgtggcctcctttaagcatgctctaaagaacggtgacatgtgtgaaacttcagataagccaaaaggcacagctagaagaaaacctgctaagaagaataaaaaccaaaagaagaatgccacagctccattgaaacagtggaaagctggtgacaaatgctctgccgtttggtcggaagatggctgcgtttacccagctaccatcacgtcagttgaccttaagagagaaacctgtgtcgtggtttatactggatatggaaacaaagaggagcaaaacctatctgatctgctttccccgacctgtgaagtagctaacaatacagaacagaacactcaggagaatgaaagccaagtttccacagacgacagtgaacactcctccagatcgctcagaagtaaagcacacagcaagtccaaagctgctccatggacctcgtttctccctccacctcccccggtgcccggggcgggattaggaccaggaaagccaggtctaaggttcagtgggccaccgccgccgccacctccccctcccccgttcttgccgtgttggatgcctccgttcccttcaggaccaccaataattcctccaccccctcccatatctcccgactgtctggatgacacggatgctctgggcagtatgctaatctcttggtacatgagtggttaccacactggttactatatgggtttcagacaaaataaaaaggagggaaagaagtgctcacatacaaattaagaagttcagctctctcccaaggagatggtttgttggtgtccctggtcgataagaacagaagtctcctcgtcacctttgtggactcttggctaagtggtgtcatcatcagggtctccctgtcccgggagtccatcctgagtcagcagcagggcatgcatagagcagcagttggaggaaccgatcaatcgatcgatcagtggcagtgtgagtgcatggaagtcagccaaactgtgactgagcacaaacggacaattgcaattttcttagaatgtcaagatttgtattaatgcctttaaaattaaataaaacccttttttgaaaaaaaaaaaaaaaaa

SEQ ID NO: 23 - protein, 634 aa, Homo sapiens [sequence omitted]

SEQ ID NO: 24 - protein, 654 aa, Mus musculus
MESPTKEIEEFESNSLKHLQPEQIEKIWLRLRGLRKYKKTSQRLRSLVKQLERGEASVVDLKKNLEYAATVLESVYIDETRRLLDTEDELSDIQSDAVPSEVRDWLASTFTRQMGMMLRRSDEKPRFKSIVHAVQAGIFVERMYRRTSNMVGLSYPPAVIDALKDVDTWSFDVFSLNEASGDHALKFIFYELLTRYDLISRFKIPISALVSFVEALEVGYSKHKNPYHNLMHAADVTQTVHYLLYKTGVANWLTELEIFAIIFSAAIHDYEHTGTTNNFHIQTRSDPAILYNDRSVLENHHLSAAYRLLQEDEEMNILVNLSKDDWREFRTLVIEMVMATDMSCHFQQIKAMKTALQQPEAIEKPKALSLMLHTADISHPAKAWDLHHRWTMSLLEEFFRQGDREAELGLPFSPLCDRKSTMVAQSQVGFIDFIVEPTFTVLTDMTEKIVSPLIDESSQTGGTGQRRSSLNSINSSDAKRSGVKSSGSDGSAPINNSVIPVDYKSFKATWTEVVQINRERWRAKVPKEEKAKKEAEEKARLAAEEKQKEMEAKSQAEQGTTSKGEKKTSGEAKSQVNGTRKGDNPRGKNSKGEKAGEKQQNGDLKDGKNKADKKDHSNTGNESKKTDGTKKRSHGSPAPSTSSTSRITLPGDYG

SEQ ID NO: 25 - protein, 768 aa, Rattus norvegicus
MTDTSHKKEGFKKCRSATFSIDGYSFTIVANEAGDKNARPLARFSRSKSQNCLWNSLIDGLTGNVKEKPRPTIVQDTRPPEEILADELPQLDSPEALVKTSFRLRSLVKQLERGEASVVDLKKNLEYAATVLESVYIDETRRLLDTEDELSDIQSDAVPSEVRDWLASTFTRQMGMMLRRSDEKPRFKSIVHAVQAGIFVERMYRRTSNMVGLSYPPAVIDALKDVDTWSFDVFSLNEASGDHALKFIFYELLTRYDLISRFKIPISALVSFVEALEVGYSKHKNPYHNLMHAADVTQTVHYLLYKTGVANWLTELEIFAIIFSAAIHDYEHTGTTNNFHIQTRSDPAILYNDRSVLENHHLSAAYRLLQEDEEMNILVNLSKDDWREFRTLVIEMVMATDMSCHFQQIKAMKTALQQPEAIEKPKALSLMLHTADISHPAKAWDLHHRWTMSLLEEFFRQGDREAELGLPFSPLCDRKSTMVAQSQVGFIDFIVEPTFTVLTDMTEKIVSPLIDETSQTGGTGQRRSSLNSINSSDAKRSGVKSSGSEGSAPINNSVIPVDYKSFKATWTEVVQINRERWRAKVPKEEKAKKEAEEKARLAAEEKQKEMEAKSQAEQGTTSKAEKKTSGETKGQVNGTRTSKGDNPRGKNSKGDKAGEKQQNGDLKDGKNKADKKDHSNTGNESKKADGTKKRSHGSPAPSTSSTSRLTLPVIKPPLRHFKRPAYASSSYAPSVPKKTDDHPVRYKMLDQRIKIKKIQNISHHWNKK

SEQ ID NO: 26 - protein, 271 aa, Homo sapiens [sequence omitted]

SEQ ID NO: 27 - protein, 271 aa, Mus musculus
MAGPQQQPPYLHLAELTASQFLEIWKHFDADGNGYIEGKELENFFQELEKARKGSGMMSKSDNFGEKMKEFMQKYDKNSDGKIEMAELAQILPTEENFLLCFRQHVGSSAEFMEAWRKYDTDRSGYIEANELKGFLSDLLKKANRPYDEPKLQEYTQTILRMFDLNGDGKLGLSEMSRLLPVQENFLLKFQGMKLTSEEFNAIFTFYDKDGSGYIDENELDALLKDLYEKNKKEMNIQQLTTYRKSVMSLAEAGKLYRKDLEIVLCSEPPV

SEQ ID NO: 28 - protein, 271 aa, Rattus norvegicus
MAGPQQQPPYLHLAELTASQFLEIWKHFDADGNGYIEGKELENFFQELEKARKGSGMMSKSDNFGEKMKEFMQKYDKNSDGKIEMAELAQILPTEENFLLCFRQHVGSSAEFMEAWRKYDTDRSGYIEANELKGFLSDLLKKANRPYDEPKLQEYTQTILRMFDLNGDGKLGLSEMSRLLPVQENFLLKFQGMKLTSEEFNAIFTFYDKDGSGYIDENELDALLKDLYEKNKKEMNIQQLTTYRKSVMSLAEAGKLYRKDLEIVLCSEPPM

SEQ ID NO: 29 - protein, 239 aa, Homo sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 30 - protein, 239 aa, Mus musculus
MPLGHIMRLDLEKIALEYIVPCLHEVGFCYLDNFLGEVVGDCVLERVKQLHYNGALRDGQLAGPRAGVSKRHLRGDQITWIGGNEEGCEAINFLLSLIDRLVLYCGSRLGKYYVKERSKAMVACYPGNGTGYVRHVDNPNGDGRCITCIYYLNKNWDAKLHGGVLRIFPEGKSFVADVEPIFDRLLFFWSDRRNPHEVQPSYATRYAMTVWYFDAEERAEAKKKFRNLTRKTESALAKD

SEQ ID NO: 31 - protein, 355 aa, Rattus norvegicus
MTLRSRRGFLSFLPGLRPPRRWLRISKRGPPTSHWASPALGGRTLHYSCRSQSGTPFSSEFQATFPAFAAKVARGPWLPQVVEPPARLSASPLCVRSGQALGACTLGVPRLGSVSEMPLGHIMRLDLEKIALEYIVPCLHEVGFCYLDNFLGEVVGDCVLERVKQLHYNGALRDGQLAGPRAGVSKRHLRGDQITWIGGNEEGCEAINFLLSLIDRLVLYCGSRLGKYYVKERSKAMVACYPGNGTGYVRHVDNPNGDGRCITCIYYLNKNWDAKLHGGVLRIFPEGKSFVADVEPIFDRLLFSWSDRRNPHEVQPSYATRYAMTVWYFDAEERAEAKKKFRNLTRKTESALAKD

SEQ ID NO: 32 - protein, 908 aa, Homo sapiens
MVCEGKRSASCPCFFLLTAKFYWILTMMQRTHSQEYAHSIRVDGDIILGGLFPVHAKGERGVPCGELKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKIIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGLERIARDSSYEQEGKVQFVIDAVYSMAYALHNMHKDLCPGYIGLCPRMSTIDGKELLGYIRAVNFNGSAGTPVTFNENGDAPGRYDIFQYQITNKSTEYKVIGHWTNQLHLKVEDMQWAHREHTHPASVCSLPCKPGERKKTVKGVPCCWHCERCEGYNYQVDELSCELCPLDQRPNMNRTGCQLIPIIKLEWHSPWAVVPVFVAILGIIATTFVIVTFVRYNDTPIVRASGRELSYVLLTGIFLCYSITFLMIAAPDTIICSFRRVFLGLGMCFSYAALLTKTNRIHRIFEQGKKSVTAPKFISPASQLVITFSLISVQLLGVFVWFVVDPPHIIIDYGEQRTLDPEKARGVLKCDISDLSLICSLGYSILLMVTCTVYAIKTRGVPETFNEAKPIGFTMYTTCIIWLAFIPIFFGTAQSAEKMYIQTTTLTVSMSLSASVSLGMLYMPKVYIIIFHPEQNVQKRKRSFKAVVTAATMQSKLIQKGNDRPNGEVKSELCESLETNTSSTKTTYISYSNHSI

SEQ ID NO: 33 - protein, 908 aa, Mus musculus
MVCEGKRSTSCPCFFLLTAKFYWILTMMQRTHSQEYAHSIRLDGDIILGGLFPVHAKGERGVPCGDLKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKIIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGLERIARDSSYEQEGKVQFVIDAVYSMAYALHNMHKELCPGYIGLCPRMVTIDGKELLGYIRAVNFNGSAGTPVTFNENGDAPGRYDIFQYQINNKSTEYKIIGHWTNQLHLKVEDMQWANREHTHPASVCSLPCKPGERKKTVKGVPCCWHCERCEGYNYQVDELSCELCPLDQRPNINRTGCQRIPIIKLEWHSPWAVVPVFIAILGIIATTFVIVTFVRYNDTPIVRASGRELSYVLLTGIFLCYSITFLMIAAPDTIICSFRRIFLGLGMCFSYAALLTKTNRIHRIFEQGKKSVTAPKFISPASQLVITFSLISVQLLGVFVWFVVDPPHTIIDYGEQRTLDPENARGVLKCDISDLSLICSLGYSILLMVTCTVYAIKTRGVPETFNEAKPIGFTMYTTCIIWLAFIPIFFGTAQSAEKMYIQTTTLTVSMSLSASVSLGMLYMPKVYIIIFHPEQNVQKRKRSFKAVVTAATMQSKLIQKGNDRPNGEVKSELCESLETNSKSSVDFQMVKSGSTS

SEQ ID NO: 34 - protein, 908 aa, Rattus norvegicus
MVCEGKRLASCPCFFLLTAKFYWILTMMQRTHSQEYAHSIRVDGDIILGGLFPVHAKGERGVPCGELKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKIIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGLERIARDSSYEQEGKVQFVIDAVYSMAYALHNMHKERCPGYIGLCPRMVTIDGKELLGYIRAVNFNGSAGTPVTFNENGDAPGRYDIFQYQINNKSTEYKIIGHWTNQLHLKVEDMQWANREHTHPASVCSLPCKPGERKKTVKGVPCCWHCERCEGYNYQVDELSCELCPLDQRPNINRTGCQRIPIIKLEWHSPWAVVPVFIAILGIIATTFVIVTFVRYNDTPIVRASGRELSYVLLTGIFLCYSITFLMIAAPDTIICSFRRIFLGLGMCFSYAALLTKTNRIHRIFEQGKKSVTAPKFISPASQLVITFSLISVQLLGVFVWFVVDPPHTIIDYGEQRTLDPENARGVLKCDISDLSLICSLGYSILLMVTCTVYAIKTRGVPETFNEAKPIGFTMYTTCIIWLAFIPIFFGTAQSAEKMYIQTTTLTVSMSLSASVSLGMLYMPKVYIIIFHPEQNVQKRKRSFKAVVTAATMQSKLIQKGNDRPNGEVKSELCESLETNTSSTKTTYISYSNHSI

SEQ ID NO: 35 - protein, 422 aa, Homo sapiens
MVSESHHEALAAPPVTTVATVLPSNATEPASPGEGKEDAFSKLKEKFMNELHKIPLPPWALIAIAIVAVLLVLTCCFCICKKCLFKKKNKKKGKEKGGKNAINMKDVKDLGKTMKDQALKDDDAETGLTDGEEKEEPKEEEKLGKLQYSLDYDFQNNQLLVGIIQAAELPALDMGGTSDPYVKVFLLPDKKKKFETKVHRKTLNPVFNEQFTFKVPYSELGGKTLVMAVYDFDRFSKHDIIGEFKVPMNTVDFGHVTEEWRDLQSAEKEEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTLQVEEEVDAMLAVKK

SEQ ID NO: 36 - protein, 421 aa, Mus musculus
MVSASRPEALAAPVTTVATLVPHNATEPASPGEGKEDAFSKLKQKFMNELHKIPLPPWALIAIAIVAVLLVVTCCFCVCKKCLFKKKNKKKGKEKGGKNAINMKDVKDLGKTMKDQALKDDDAETGLTDGEEKEEPKEEEKLGKLQYSLDYDFQNNQLLVGIIQAAELPALDMGGTSDPYVKVFLLPDKKKKFETKVHRKTLNPVFNEQFTFKVPYSELGGKTLVMAVYDFDRFSKHDIIGEFKVPMNTVDFGHVTEEWRDLQSAEKEEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTLQVEEEVDAMLAVKK

SEQ ID NO: 37 - protein, 421 aa, Rattus norvegicus
MVSASHPEALAAPVTTVATLVPHNATEPASPGEGKEDAFSKLKQKFMNELHKIPLPPWALIAIAIVAVLLVVTCCFCVCKKCLFKKKNKKKGKEKGGKNAINMKDVKDLGKTMKDQALKDDDAETGLTDGEEKEEPKEEEKLGKLQYSLDYDFQNNQLLVGIIQAAELPALDMGGTSDPYVKVFLLPDKKKKFETKVHRKTLNPVFNEQFTFKVPYSELGGKTLVMAVYDFDRFSKHDIIGEFKVPMNTVDFGHVTEEWRDLQSAEKEEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTLQVEEEVDAMLAVKK

SEQ ID NO: 38 - protein, 486 aa, Homo sapiens
MYIKMATLANGQADNASLSTNGLGSSPGSAGHMNGLSHSPGNPSTIPMKDHDAIKLFIGQIPRNLDEKDLKPLFEEFGKIYELTVLKDRFTGMHKGCAFLTYCERESALKAQSALHEQKTLPGMNRPIQVKPADSESRGGSSCLRQPPSQDRKLFVGMLNKQQSEDDVRRLFEAFGNIEECTILRGPDGNSKGCAFVKYSSHAEAQAAINALHGSQTMPGASSSLVVKFADTDKERTMRRMQQMAGQMGMFNPMAIPFGAYGAYAQALMQQQAALMASVAQGGYLNPMAAFAAAQMQQMAALNMNGLAAAPMTPTSGGSTPPGITAPAVPSIPSPIGVNGFTGLPPQANGQPAAEAVFANGIHPYPAQSPTAADPLQQAYAGVQQYAGPAAYPAAYGQISQAFPQPPPMIPQQQREGPEGCNLFIYHLPQEFGDAELMQMFLPFGFVSFDNPASAQTAIQAMNGFQIGMKRLKVQLKRPKDANRPY

SEQ ID NO: 39 - protein, 504 aa, Mus musculus
MYIKMATLANGQADNASLSTNGLGSSPGSAGHMNGLSHSPGNPSTIPMKDHDAIKLFIGQIPRNLDEKDLKPLFEEFGKIYELTVLKDRFTGMHKGCAFLTYCERESALKAQSALHEQKTLPGMNRPIQVKPADSESRGGSSCLRQPPSHRKLFVGMLNKQQSEDDVRRLFEAFGNIEECTILRGPDGNSKGCAFVKYSSHAEAQAAINALHGSQTMPGASSSLVVKFADTDKERTMRRMQQMAGQMGMFNPMAIPFGAYGAYAQALMQQQAALMASVAQGGYLNPMAAFAAAQMQQMAALNMNGLAAAPMTPTSGGSTPPGITAPAVPSIPSPIGVNGFTGLPPQANGQPAAEAVFANGIHPYPAQSPTAADPLQQAYAGVQQYAGPAYPAAYGQISQAFPQPPPMIPQQQREGPEGCNLLIYHLPQEFGDAELMQMFLPFGNVISSKVFVDRATNQSKCFGFVSFDNPASAQTAIQAMNGFQIGMKRLKVQLKRPKDANRPY

SEQ ID NO: 40 - protein, 329 aa, Rattus norvegicus
MLNKQQSEDDVRRLFEAFGNIEECTILRGPDGNSKGCAFVKYSSHAEAQAAINALHGSQTMPGASSSLVVKFADTDKERTMRRMQQMAGQMGMFNPMAIPFGAYGAYAQALMQQQAALMASVAQGGYLNPMAAFAAAQMQQMAALNMNGLAAAPMTPTSGGSTPPGITAPAVPSIPSPIGVNGFTGLPPQANGQPAAEAVFANGIHPYPAQSPTAADPLQQAYAGVQQYAGPAAYPAAYGQISQAFPQPPPMIPQQQREGPEGCNLLIYHLPQEFGDAELMQMFLPFGFVSFDNPASAQTAIQAMNGFQIGMKRLKVQLKRPKDANRPY

SEQ ID NO: 41 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 42 - protein, 288 aa, Mus musculus
MAMGSGGAGSEQEDTVLFRRGTGQSDDSDIWDDTALIKAYDKAVASFKHALKNGDICETPDKPKGTARRKPAKKNKSQKKNATTPLKQWKVGDKCSAVWSEDGCIYPATITSIDFKRETCVVVYTGYGNREEQNLSDLLSPTCEVANSTEQNTQENESQVSTDDSEHSSRSLRSKAHSKSKAAPWTSFLPPPPPMPGSGLGPGKPGLKFNGPPPPPPLPPPPFLPCWMPPFPSGPPIIPPPPPISPDCLDDTDALGSMLISWYMSGYHTGYYMGFRQNKKEGKCSHTN

SEQ ID NO: 43 - protein, 288 aa, Rattus norvegicus
MAMGSGGAGSEQEDTVLFRRGTGQSDDSDIWDDTALIKAYDKAVASFKHALKNGDMCETSDKPKGTARRKPAKKNKNQKKNATAPLKQWKAGDKCSAVWSEDGCVYPATITSVDLKRETCVVVYTGYGNKEEQNLSDLLSPTCEVANNTEQNTQENESQVSTDDSEHSSRSLRSKAHSKSKAAPWTSFLPPPPPVPGAGLGPGKPGLRFNGPPPPPPPPPPFLPCWMPPFPSGPPIIPPPPPISPDCLDDTDALGSMLISWYMSGYHTGYYMGFRQNKKEGKKCSHTN

(19) United States
(12) Patent Application Publication
(10) Pub. No.: US 2016/0045617 A1
(43) Pub. Date: Feb. 18, 2016

(54) TREATMENT OF PROXIMAL SPINAL MUSCULAR ATROPHY

(71) Applicants: Justin C. Lee, New York, NY (US); Christopher Henderson, New York, NY (US)

(72) Inventors: Justin C. Lee, New York, NY (US); Christopher Henderson, New York, NY (US)

(21) Appl. No.: 14/783,034

(22) PCT Filed: Apr. 10, 2014
(86) PCT No.: PCT/US14/33585

§ 371 Date: Oct. 7, 2015

Related U.S. Application Data
(60) Provisional application No. 61/810,628, filed on Apr. 10, 2013.

(51) Int. Cl. A61K 48/00; A61K 38/13; A61K 31/7088; A61K 38/10; A61K 31/663; A61K 45/06

(52) U.S. Cl. CPC A61K 48/00; A61K 31/663; A61K 45/06; A61K 31/7088; A61K 38/10; A61K 38/13

(57) ABSTRACT

The present invention provides, inter alia, methods and pharmaceutical compositions for treating or ameliorating an effect of proximal spinal muscular atrophy (SMA) and methods for preventing or slowing motor neuron death in a subject having SMA. The methods include administering to a subject in need thereof a modulator of a gene selected from the group consisting of phosphodiesterase 1c (Pde1c), Calbindin 2 (Calb2), Egl nine homolog 3 (Eg13), Metabotropic glutamate receptor 8 (mGluR8), Syn aptotagmin 1 (Syt1), CUGBP, Elav-like family member 4 (Celf4), and combinations thereof in an amount effective to treat or ameliorate an effect of SMA. Also provided are methods for preventing or slowing motor neuron death.

### CROSS-REFERENCE TO RELATED APPLICATIONS

The present invention claims benefit to U.S. provisional application Ser. No. 61/810,628 filed Apr. 10, 2013, the entire contents of which are incorporated by reference as if recited herein.

### FIELD OF INVENTION

The present invention provides, inter alia, methods and pharmaceutical compositions for treating or ameliorating an effect of proximal spinal muscular atrophy (SMA). Methods for preventing or slowing motor neuron death are also provided.

### INCORPORATION BY REFERENCE OF SEQUENCE LISTING

This application contains references to amino acids and/or nucleic acid sequences that have been filed concurrently herewith as sequence listing text file “0364779.txt”, file size of 146 KB, created on Apr. 10, 2014. The aforementioned sequence listing is hereby incorporated by reference in its entirety pursuant to 37 C.F.R. §1.52(e)(5).

### BACKGROUND OF THE INVENTION

Proximal spinal muscular atrophy (SMA) is a pediatric neuromuscular disease characterized by widespread loss of motor neurons and death from respiratory failure by two years of age in severely affected patients. Despite this, patients retain oculomotor and external sphincter function. Moreover, the sparing of the diaphragm in conjunction with severe recession of the intercostal muscles produces a “bell-shaped chest” that is pathognomonic for SMA. There are currently no effective treatments for this disorder.

Most therapeutic strategies have focused on correcting the reduced expression of smooth muscle neurons (SMN), which is the cause of the disease. Several promising approaches to this using antisense oligonucleotides or small-molecule regulators are being, or soon will be, evaluated in the clinic. However, it remains a real possibility that SMN modulation alone will not be sufficient to completely overcome disease symptoms, or that the agents used for SMN modulation will prove to have problems.

Accordingly, there is a need for other, novel candidate therapeutic targets to complement or provide an alternative to the SMN-focused strategies. This invention is directed to meeting these and other needs.

### SUMMARY OF THE INVENTION

One embodiment of the present invention is a method for treating or ameliorating an effect of proximal spinal muscular atrophy (SMA). This method comprises administering to a subject in need thereof a modulator of a gene selected from the group consisting of phosphodiesterase 1c (Pde1c), Calbindin 2 (Calb2), Egl nine homolog 3 (Egl3), Metabotropic glutamate receptor 8 (mGluR8), Synaptotagmin 1 (Syt1), CUGBP, Elav-like family member 4 (Celf4), and combinations thereof in an amount effective to treat or ameliorate an effect of SMA.

Another embodiment of the present invention is a method for preventing or slowing motor neuron death in a subject having proximal spinal muscular atrophy (SMA). This method comprises administering to the subject a modulator of a gene selected from the group consisting of Pde1c, Calb2, Egl3, mGluR8, Syt1, Celf4, and combinations thereof in an amount effective to prevent or slow motor neuron death in the subject.

An additional embodiment of the present invention is a pharmaceutical composition for treating or ameliorating an effect of proximal spinal muscular atrophy (SMA) in a subject in need thereof. This pharmaceutical composition comprises a pharmaceutically acceptable carrier or diluent and an amount of a modulator of a gene selected from the group consisting of Pde1c, Calb2, Egl3, mGluR8, Syt1, Celf4, and combinations thereof, which amount is effective to treat or ameliorate an effect of SMA in the subject.

A further embodiment of the present invention is a method for preventing or slowing motor neuron death. This method comprises contacting a motor neuron with a modulator of a gene selected from the group consisting of Pde1c, Calb2, Egl3, mGluR8, Syt1, Celf4, and combinations thereof in an amount effective to prevent or slow motor neuron death.

### DETAILED DESCRIPTION OF THE INVENTION

One embodiment of the present invention is a method for treating or ameliorating an effect of proximal spinal muscular atrophy (SMA). This method comprises administering to a subject in need thereof a modulator of a gene selected from the group consisting of Pde1c, Calb2, Egl3, mGluR8, Syt1, Celf4, and combinations thereof in an amount effective to treat or ameliorate an effect of SMA.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and pharmaceutical compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject, e.g., patient, population. Accordingly, a given subject or subject, e.g., patient, population may fail to respond or respond inadequately to treatment.

As used herein, the terms “ameliorate,” “ameliorating,” and grammatical variations thereof mean to decrease the severity of one or more symptoms of the particular condition or disease, e.g., SMA, in a subject.

As used herein, a “subject” is a mammal, preferably, a human. In addition to humans, categories of mammals within the scope of the present invention include, for example, agricultural animals, domestic animals, laboratory animals, etc. Some examples of agricultural animals include cows, pigs, horses, goats, etc. Some examples of domestic animals include dogs, cats, etc. Some examples of laboratory animals include rats, mice, rabbits, guinea pigs, etc.

As used herein, the term “gene” includes a nucleic acid sequence that when translated, transcribed, and otherwise processed (such as post-transcriptional or post-translational processing) that results in a protein or polypeptide. The term “gene”, as used herein, also includes gene products, such as transcribed mRNA of the gene and/or the resultant protein/polypeptide. It is further noted that certain genes, such as Pde1c, may be alternatively spliced, thus producing different isoforms of the protein, and in the case of Pde1c, more than 10 human isoforms have been identified (Bolger, 2006). As with the convention in this field, the recitation of the gene, such as Pde1c, includes all of the isoforms as well. Non-limiting examples of Pde1c are depicted in SEQ ID NOs: 2-4 and 23-25. Non-limiting examples of Calb2 are depicted in SEQ ID NOs: 5-7 and 26-28. Non-limiting examples of Egl3 are depicted in SEQ ID NOs: 8-10 and 29-31. Non-limiting examples of mGluR8 are depicted in SEQ ID NOs: 11-13 and 32-34. Non-limiting examples of Syt1 are depicted in SEQ ID NOs: 14-16 and 25-37. Non-limiting examples of Celf4 are depicted in SEQ ID NOs: 17-19 and 38-40.

As used herein, the term “modulator” means an agent that elicits an effect on gene expression or protein activity level. For example, in one aspect of this embodiment, the modulator is an inhibitor of a gene selected from the group consisting of Pde1c, Calb2, Egl3, and combinations thereof. As used herein, an “inhibitor” means an agent that reduces or suppresses gene expression, the amount of protein, or protein activity.

Inhibitors of Pde1c include, but are not limited to, zaprinast, 8-methoxymethyl-1-methyl-3-(2-methylpropyl)xanthine (8MM-IBMX), vinpocetine, 3-isobutyl-1-methylxanthine (IBMX), SCH51866 (Merck & Co., Whitehouse Station, N.J.), Compound 30 (Merck & Co.), Compound 31 (Merck & Co.),

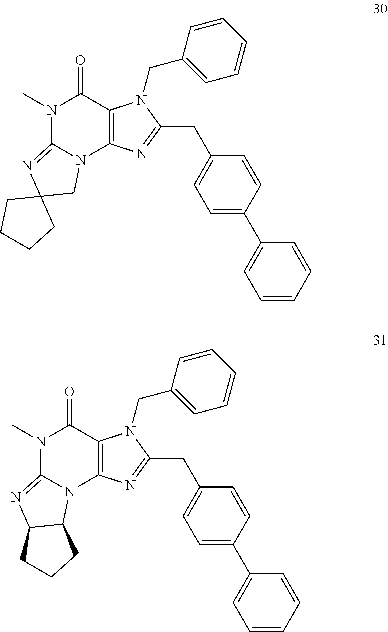 [SMILES: CN1C(=O)c2c(nc(Cc3ccc(-c4ccccc4)cc3)n2Cc2ccccc2)N2CC3(CCCC3)N=C12 ; CN1C(=O)c2c(nc(Cc3ccc(-c4ccccc4)cc3)n2Cc2ccccc2)N2C1=N[C@@H]1CCC[C@@H]12]

nimodipine, IC86340 (Keravis et al., 2012; Nagel et al., 2006), IC295 (Keravis et al., 2012; Vandeput et al., 2007), IC224 (Eli Lilly, Indianapolis, Ind.), dioclein, KS505a, DIF-1, EGTA, trifluoroperazine, W7, sildenafil, vardenafil, amantadine, deprenyl, ginsenoids, theophylline, HFV-1017 (Gaiichi Sankyo, Parsippany, N.J.), ITI-214 (Intra-Cellular Therapies Inc., New York, N.Y.), K-259-2 (Kyowa Hakko Kirin Pharma Inc., Princeton, N.J.), KS-501 (Kyowa Hakko Kirin Pharma Inc.), KS-505 (Kyowa Hakko Kirin Pharma Inc.), KS-619-1 (Kyowa Hakko Kirin Pharma Inc), Sch-45752 (Merck & Co., Whitehouse Station, N.J.), Sch-59498 (Merck & Co), and CV-159 (Mitsubishi Tanabe Pharma, Jersey City, N.J.), as well as derivatives of 1-methyl-3-isobutylxanthine:

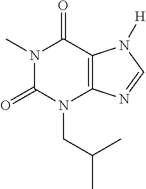 [SMILES: CC(C)Cn1c(=O)n(C)c(=O)c2[nH]cnc21]

with substitutions consisting of a moiety at positions 2 and 8 independently selected from the group consisting of an alkyl (C₁ to C₃), a flouroalkyl (F₁ to F₃), a chloroalkyl (Cl₁ to Cl₃), an aryl (C₅ to C₆), a fluoroaryl (F₁ to F₂), and a chloroaryl (Cl₁ to Cl₂), as disclosed in U.S. Pat. No. 6,812,239. In one preferred embodiment, the inhibitor of Pde1c is ITI-214.

Inhibitors of Calb2 include, but are not limited to, cyclosporin A and a dodecapeptide of the sequence ISSIKEKYPSHS (SEQ ID NO. 1), or a contiguous fragment thereof, in which up to three amino acid residues are replaced, such as those disclosed by U.S. Patent Application Publication No. 2011/0076347.

Inhibitors of Egl3 include, but are not limited to, antianaemic siRNA therapy (Alnylam Pharmaceuticals, Cambridge, Mass.), iron chelators, dimethyloxaloglycine (DMOG), synthetic 2-oxogluturate antagonists, iron-displacing metals, malonic acid, 3-nitroproprionic acid, theonyl trifluoracetone, 2-oxoglutarate analogs (including N-oxalylglycine, N-oxalyl-2S-alanine, and N-oxalyl-2R-alanine, and other analogs disclosed in U.S. Patent Application Publication No. 2010/0016434), benzimidazol-4-ylcarboxamide derivatives disclosed in U.S. Patent Application Publication No. 2011/0039895, pyrimidinedione N-substituted glycine derivatives disclosed in U.S. Patent Application Publication No. 2011/0144167, pyridazinedione N-substituted glycine derivatives disclosed in U.S. Pat. No. 7,608,621, 4-ox-2-thioxo-1,2,3,4-tetrahydro-7-quinazolinecarboxamide derivatives disclosed in U.S. Patent Application Publication No. 2010/0298324, and those Egl3 inhibitor compounds and compositions disclosed in U.S. Patent Application Publication Nos. 2011/0111058 and 2012/0121720.

In another aspect of this embodiment, the modulator is an activator of a gene selected from the group consisting of mGluR8, Syt1, Celf4, and combinations thereof. As used herein, “activator” means any agent that increases gene expression, the amount of protein, or protein activity level.

Activators of mGluR8 include, but are not limited to, L-glutamic acid, cysteine, and amino acid derivatives such as (S)-3,4-dicarboxyphenylglycine ((S)-3,4-DCPG), (RS)-4-phosphonophenylglycine, and L-serine-O-phosphate, as well as L-2-amino-4 phosphonobutyrate.

Activators of Syt1 include, but are not limited to, phosphatidylinositol polyphosphates, such as phosphatidylinositol 4,5-bisphosphate.

Activators of CELF4 include various CELF4 nucleic acids and polypeptides, including CELF4 Δ5.1, CELF4 Δ5.2, CELF4 (+48), CELF4 Δ3.1, CELF4 Δ3.2, CELF4 Δ3.3, CELF4 Δ3.4, CELF4.24, CELF4 DD1, CELF4 DD2, and CELF4 DD3, as disclosed in Singh et al. (2004).

In an additional aspect of this embodiment, the method further comprises co-administering to the subject a modulator of survival motor neuron (SMN) expression.

In the present invention, two or more modulators may be administered to a subject together in the same composition, simultaneously in separate compositions, or as separate compositions administered at different times, as deemed most appropriate by a physician.

Preferably, the modulator of SMN expression causes an increase in SMN expression. More preferably, the modulator of SMN expression is selected from the group consisting of a wild type SMN-1 gene for use in gene therapy, a small molecule, and an antisense oligonucleotide.

As used herein, the term “wild type” refers to that version of a gene most commonly found in nature. Examples of wild type SMN-1 genes are depicted in SEQ ID Nos. 20-22.

As used herein, the term “gene therapy” refers to any procedure that uses nucleic acids to heal, cure, or otherwise improve a condition in a subject. In gene therapy, nucleic acids need to be delivered into specific cells. Delivery methods include viral and non-viral means, which are known in the art. E.g., Patil et al., AAPS J. 7(1): E61-E77 (2005); Gascón et al., Non-Viral Delivery Systems in Gene Therapy (2013); Somiari et al., Molecular Therapy, 2(3), 178-187 (2000); Herweijer, H., and J. A. Wolff, Gene therapy 10(6): 453-458 (2003); and Nayerossadat et al., Advanced biomedical research 1(2):1-11 (2012).

Viral means for delivering gene therapy involve the use of viral vectors. Viral vectors are genetically modified viruses that can carry a therapeutic genetic payload and have been reprogrammed to allow for infection and subsequent transmittal of said payload into specific tissues without the side effects typically associated with wild-type viral infection. A number of viruses can be used as viral vectors, including retroviruses, adenoviruses, herpes simplex virus, lentiviruses, Poxvirus, and Epstein-Barr virus. While safer than wild-type viruses, viral vectors may induce an immune response, occasionally necessitating the use of non-viral delivery methods.

Non-viral delivery methods include, but are not limited to, physical methods, such as injection of naked DNA, electroporation, gene gun bombardment, and ultrasound, as well as biochemical methods. Another delivery technique, magnetofection, combines physical and biochemical elements.

Introduction of naked DNA may be achieved via intradermal, intramuscular, and intravascular injection means. Though injection of naked DNA alone leads to low levels of transfection, electroporation can be used in conjunction with injection of naked DNA to increase transfection efficiency. Intravascular methods may be performed systemically or regionally depending on the point of injection. For example, high levels of expression in hepatocytes can be achieved via injection of naked DNA into the tail vein of mice. Naked DNA can also be injected into the afferent or efferent vessel of the liver in monkeys, yielding a similar result. Intravascular delivery of naked DNA to specific regions of skeletal muscle can be improved through the use of catheters and tourniquets.

Other physical methods of delivery transiently facilitate DNA entry into a cell. Gene gun bombardment involves the acceleration of micron-sized metal particles (e.g. gold) coated with DNA into cells by pressurized gas. Pores in the cell membrane result, allowing for delivery of the DNA payload and subsequent gene expression in surface epithelia (e.g. skin, surgically exposed regions). Ultrasound can also induce pore formation and DNA penetration, but can access internal organs and tumors without surgery.

Biochemical methods typically involve the use of cationic particles in packaging negatively-charged nucleic acids for delivery. The resulting cationic complexes are incorporated into the cell by endocytosis and the nucleic acids are subsequently released to the nucleus. Cationic particles include, but are not limited to, monovalent cationic lipids, polyvalent cationic lipids, guanidine-containing lipids, cholesterol derivatives, cationic polymers such as poly(ethylenimine) (PEI), poly-L-lysine (PLL), and protamine, and lipid-polymer hybrids.

Magnetofection is a hybrid physical/biochemical technique that utilizes nucleic acids conjugated to cation-coated magnetic nanoparticles. Application of an appropriate magnetic field allows for the concentration of nucleic acid-magnetic nanoparticle complexes on the surface of cells, yielding high transfection efficiencies. An appropriate magnetic field also has the advantage of restricting the complexes to a specific region of the body, reducing systemic exposure to the treatment.

Small molecule modulators of SMN expression include, but are not limited to, indoprofen, prolactin, phenylbutyrate, and trichostatin A.

Examples of antisense oligonucleotides useful for modulating SMN expression include the exon8-hnRNPA1 bifunctional RNA, as disclosed by Dickson et al., 2008.

Another embodiment of the present invention is a method for preventing or slowing motor neuron death in a subject having proximal spinal muscular atrophy (SMA). This method comprises administering to the subject a modulator of a gene selected from the group consisting of Pde1c, Calb2, Egl3, mGluR8, Syt1, Celf4, and combinations thereof in an amount effective to prevent or slow motor neuron death in the subject. Suitable subjects are as set forth above.

As used herein, the terms “prevent”, “preventing” and grammatical variations thereof mean to keep, e.g., motor neuron death, from happening. As used herein, the terms “slow”, “slowing” and grammatical variations thereof mean to delay, e.g., motor neuron death.

In one aspect of this embodiment, the modulator is an inhibitor of a gene selected from the group consisting of Pde1c, Calb2, Egl3, and combinations thereof. Suitable inhibitors are as set forth above.

In another aspect of the present embodiment, the modulator is an activator of a gene selected from the group consisting of mGluR8, Syt1, Celf4, and combinations thereof. Suitable activators are as set forth above.

In an additional aspect of the present embodiment, the method further comprises co-administering to the subject a modulator of SMN expression. Suitable and preferred modulators of SMN expression are as disclosed herein.

An additional embodiment of the present invention is a pharmaceutical composition for treating or ameliorating an effect of proximal spinal muscular atrophy (SMA) in a subject in need thereof. This pharmaceutical composition comprises a pharmaceutically acceptable carrier or diluent and an amount of a modulator of a gene selected from the group consisting of Pde1c, Calb2, Egl3, mGluR8, Syt1, Celf4, and combinations thereof, which amount is effective to treat or ameliorate an effect of SMA in the subject. Suitable subjects are as set forth above.

In one aspect of this embodiment, the modulator is an inhibitor of a gene selected from the group consisting of Pde1c, Calb2, Egl3, and combinations thereof. Suitable inhibitors are as set forth above.

In another aspect of this embodiment, the modulator is an activator of a gene selected from the group consisting of mGluR8, Syt1, Celf4, and combinations thereof. Suitable activators are as set forth above.

In an additional aspect of this embodiment, the pharmaceutical composition further comprises a modulator of SMN expression. Suitable and preferred modulators of SMN expression are as disclosed herein.

A further embodiment of the present invention is a method for preventing or slowing motor neuron death. This method comprises contacting a motor neuron with a modulator of a gene selected from the group consisting of Pde1c, Calb2, Egl3, mGluR8, Syt1, Celf4, and combinations thereof in an amount effective to prevent or slow motor neuron death.

In one aspect of this embodiment, the modulator is an inhibitor of a gene selected from the group consisting of Pde1c, Calb2, Egl3, and combinations thereof. Suitable inhibitors are as set forth above.

In another aspect of this embodiment, the modulator is an activator of a gene selected from the group consisting of mGluR8, Syt1, Celf4, and combinations thereof. Suitable activators are as set forth above.

In an additional aspect of this embodiment, the method further comprises contacting the motor neuron with a modulator of SMN expression. Suitable and preferred modulators of SMN expression are as disclosed herein.

In another aspect of this embodiment, the motor neuron is a mammalian motor neuron. Preferably, the motor neuron is a human motor neuron.

In this embodiment, contacting includes administration of the modulator to a subject as defined herein, preferably a human patient, as well as delivery of the modulator to, e.g., motor neurons in vitro, such as in a tissue culture container.

In the present invention, an “effective amount” of a modulator disclosed herein is that amount of such modulator that is sufficient to achieve beneficial or desired results as described herein when administered to a subject or in vitro to motor neuron cells. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a modulator according to the invention will be that amount of the modulator, which is the lowest dose effective to produce the desired effect.

A suitable, non-limiting example of a dosage of modulator disclosed herein is from about 1 mg/kg to about 2400 mg/kg per day, such as from about 1 mg/kg to about 1200 mg/kg per day, including from about 50 mg/kg to about 1200 mg/kg per day. Other representative dosages of such modulators include about 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. The effective dose of the modulators disclosed herein maybe administered as two, three, four, five, six or more sub-doses, administered separately at appropriate intervals throughout the day.

A pharmaceutical composition of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a pharmaceutical composition of the present invention may be administered in conjunction with other treatments. A pharmaceutical composition of the present invention maybe encapsulated or otherwise protected against gastric or other secretions, if desired.

The pharmaceutical compositions of the invention comprise one or more active ingredients in admixture with one or more pharmaceutically acceptable carriers or diluents and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable carriers or diluents are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier or diluent used in a pharmaceutical composition of the invention must be “acceptable” in the sense of being compatible with the other ingredients of the formulation and not injurious to the subject. Carriers or diluents suitable for a selected dosage form and intended route of administration are well known in the art, and acceptable carriers or diluents for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers or diluents and, optionally, one or more fillers, extenders, binders, humectants, disintegrating agents, solution retarding agents, absorption accelerators, wetting agents, absorbents, lubricants, and/or coloring agents. Solid pharmaceutical compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These pharmaceutical compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions of the present invention for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions of the present invention which are suitable for vaginal administration also include pessaries, tampons, creams, gels, pastes, foams or spray formulations containing such pharmaceutically-acceptable carriers or diluents as are known in the art to be appropriate.

Dosage forms for topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier or diluent. The ointments, pastes, creams and gels may contain excipients. Powders and sprays may contain excipients and propellants.

Pharmaceutical compositions of the present invention suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsulated matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above. Kits containing one or more doses of the pharmaceutical compositions of the present invention alone or as part of a combination therapy are also within the scope of the present invention.

### Additional Definitions

### Nucleic Acid

“Nucleic acid” or “oligonucleotide” or “polynucleotide” used herein mean at least two nucleotides covalently linked together.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods.

The nucleic acid may also be a RNA such as a mRNA, tRNA, antisense RNA (asRNA), short hairpin RNA (shRNA), short interfering RNA (sRNA), double-stranded RNA (dsRNA), transcriptional gene silencing RNA (ptgsRNA), Piwi-interacting RNA, pri-miRNA, pre-miRNA, micro-RNA (miRNA), or anti-miRNA, as described, e.g., in U.S. patent application Ser. Nos. 11/429,720, 11/384,049, 11/418,870, and 11/429,720 and Published International Application Nos. WO 2005/116250 and WO 2006/126040.

An asRNA is a single-stranded RNA molecule with a nucleotide sequence complementary to a sense strand RNA, i.e., messenger RNA. Antisense RNA may be introduced into a cell to inhibit translation of a complementary mRNA by base pairing to it and physically obstructing the translation machinery.

sRNA gene-targeting may be carried out by transient sRNA transfer into cells, achieved by such classic methods as lipid-mediated transfection (such as encapsulation in liposome, complexing with cationic lipids, cholesterol, and/or condensing polymers, electroporation, or microinjection). sRNA gene-targeting may also be carried out by administration of sRNA conjugated with antibodies or sRNA complexed with a fusion protein comprising a cell-penetrating peptide conjugated to a double-stranded (ds) RNA-binding domain (DRBD) that binds to the sRNA (see, e.g., U.S. Patent Application Publication No. 2009/0093026).

An shRNA molecule has two sequence regions that are reversely complementary to one another and can form a double strand with one another in an intramolecular manner. shRNA gene-targeting may be carried out by using a vector introduced into cells, such as viral vectors (lentiviral vectors, adenoviral vectors, or adeno-associated viral vectors for example). The design and synthesis of siRNA and shRNA molecules are known in the art, and may be commercially purchased from, e.g., Gene Link (Hawthorne, N.Y.), Invitrogen Corp. (Carlsbad, Calif.), Thermo Fisher Scientific, and Dharmacon Products (Lafayette, Colo.).

The nucleic acid may also be an aptamer, an intramer, or a spiegelmer. The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), disclosed in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′—OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those disclosed in U.S. Pat. Nos. 5,235,033 and 5,034,506. Nucleic acids containing one or more non-naturally occurring or modified nucleotides are also included within the definition of nucleic acid. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as disclosed in Krutzfeldt et al., Nature (Oct. 30, 2005), Soutschek et al., Nature 432:173-178 (2004), and U.S. Patent Application Publication No. 20050107325. Modified nucleotides and nucleic acids may also include locked nucleic acids (LNA), as disclosed in U.S. Patent Application Publication No. 20020115080. Additional modified nucleotides and nucleic acids are disclosed in U.S. Patent Application Publication No. 20050182005. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

### Peptide, Polypeptide, Protein

The terms “peptide,” “polypeptide,” and “protein” are used interchangeably herein. In the present invention, these terms mean a linked sequence of amino acids, which may be natural, synthetic, or a modification, or combination of natural and synthetic. The term includes antibodies, antibody mimetics, domain antibodies, lipocalins, targeted proteases, and polypeptide mimetics. The term also includes vaccines containing a peptide or peptide fragment intended to raise antibodies against the peptide or peptide fragment.

### Small Molecule

The phrase “small molecule” includes any chemical or other moiety, other than polysaccharides, polypeptides, and nucleic acids, that can act to affect biological processes. Small molecules can include any number of therapeutic agents presently known and used, or can be synthesized in a library of such molecules for the purpose of screening for biological function(s). Small molecules are distinguished from macromolecules by size. The small molecules of this invention usually have a molecular weight less than about 5,000 daltons (Da), preferably less than about 2,500 Da, more preferably less than 1,000 Da, most preferably less than about 500 Da.

As used herein, preferably, the small molecule is an organic compound, which refers to any carbon-based compound other than biologics such as nucleic acids, polypeptides, and polysaccharides. In addition to carbon, organic compounds may contain calcium, chlorine, fluorine, copper, hydrogen, iron, potassium, nitrogen, oxygen, sulfur and other elements. An organic compound may be in an aromatic or aliphatic form.

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

The terminology used herein is for the purpose of describing particular embodiments only and is not intended to be limiting. As used in the specification and the appended claims, the singular forms “a,” “an,” and “the” include plural referents unless the context clearly dictates otherwise.

The following examples are provided to further illustrate the methods and compositions of the present invention. These examples are illustrative only and are not intended to limit the scope of the invention in any way.

### EXAMPLES

### Example 1

In the present invention, the general approach was to understand why certain neuronal populations are more or less resistant to the disease, e.g., SMA, and to use this information to identify potential disease modifiers, and therefore candidate targets.

We performed an extensive characterization of differential motor pool susceptibility in the SMN delta7 mouse model of SMA (The Jackson Laboratory, Bar Harbor, Me.) (Le et al., 2005), as well as human autopsies from Type I patients and a control. The characterization was done through studies in normal wildtype mice, but based on information about the SMA pathology we gained from studying mouse models and human post mortem tissue. We used wild type mice for RNA isolation and gene expression analyses in the eleven different motor pools. Our studies characterizing motor neuron pathology and differential vulnerability in the SMN delta7 mice used littermates that were heterozygous or homozygous for the Smn1 gene (these mice have normal levels of SMN protein and are indistinguishable from wild type mice). Our studies of human pathology used autopsy samples from a seven month-old patient with a congenital diaphragmatic hernia as a control. Findings between mouse and human SMA were highly concordant, providing confidence that we can gain insights into disease vulnerability in human patients from studying the mouse.

We isolated RNA from at least eleven differentially vulnerable motor neuron pools in normal mice. The SMA-resistant motor neuron pools were: phrenic, abducens, superior oblique, superior rectus, oculomotor (including medial rectus, inferior rectus, inferior oblique), and sternohyoid. The SMA-vulnerable motor neuron pools were: biventer cervicis, masseter, triceps, intercostals, flexor digitorum brevis 2/3. Individual muscles were labeled with fluorophore-conjugated cholera toxin B, and and laser capture microdissection of individual labeled motor neurons was performed, as described in more detail below.

### Retrograde Labeling of Individual Motor Pools.

Conjugated cholera toxin B (Invitrogen, Life Technologies, Grand Island, N.Y.) was prepared according to the manufacturer's protocol. Mouse pups were anesthetized with isoflurane (5% induction, 1% maintenance). Then, a minimal incision necessary to expose the muscle of interest was made in each pup, and 1-3 μl of fluorophore-conjugated cholera toxin B, depending on the size of the muscle, was injected. About 3 ml of Phosphate-Buffered Saline (PBS) was pipetted onto the muscle to wash away any leaked CTB. The incision was then closed with Vetbond (Thermo Fisher Scientific Inc., Waltham, Mass.).

After the surgery, the pups were returned to the mothers. After 48 hours, the pups were sacrificed using CO₂ asphyxiation. The spinal cord segment that contained the motor pool of interest was then carefully dissect out from each pup and embed in OCT media on dry ice.

The injected muscles were dissected to ensure specificity of injection. Further steps were taken only if fluorescence was limited to the desired muscle.

Each spinal cord was cryosectioned onto a MembraneSlide 1.0 PEN (Zeiss, Oberkochen, Germany) at 10 μm, and the slides were stored at −80° C. until use.

### Laser Capture Microdissection (LCM) (Leica LMD 6500):

The lysis buffer (from Stratagene Absolutely RNA Nanoprep kit, Agilent Technologies, Santa Clara, Calif.) was prepared by adding 0.7 μl of 14.2M β-mercaptoethanol (provided with kit) per 100 μl of lysis buffer, for a final concentration of 0.1M β-mercaptoethanol. The membrane slides were kept on dry ice and removed individually for LCM.

Prior to LCM, the slide of interest was removed, and any condensation on the slide was allowed to dry (approximately 3-5 minutes). Then, about 40 μl of lysis buffer (with β-mercaptoethanol) were pipetted into the cap of the collection tube and loaded onto the LCM apparatus. Labeled motor neurons in the ventral horn of the spinal cord were identified, and individual cells of the motor pool were cut out and placed into into a collection cap. Finally, the collection tube was placed on dry ice and stored at −80° C. until the RNA isolation step.

### RNA Isolation

RNA were isolated using the Absolutely RNA Nanoprep Kit (Agilent Technologies). The quantity and quality of RNA were analyzed with Agilent Bioanalyzer 2100.

The strategy set forth above allowed us to determine gene differences that are specific to motor neurons within the molecular heterogenity of the ventral spinal cord. Moreover, analyzing a large number of motor neuron pools that are paired at different levels of the neuraxis should eliminate gene differences that are related to development, anatomical identity, or presynaptic connectivity and highlight genes that may be related to disease vulnerability. Transcriptional analysis on SMA-vulnerable and SMA-resistant motor pools identified 37 genes that are significantly differentailly regulated (p<0.0005) and correlate very tightly with disease vulnerability in SMA.

This study indicates that the top candidate vulnerability genes include Pde1c, p<1.7×10⁻⁷; Calbindin 2 (Calb2), p<2.1×10⁻⁴; and Egl nine homolog 3 (Egl3), p<4.1×10⁻⁴. Top candidate disease resistance genes include metabotropic glutamate receptor 8 (mGluR8), p<4.4×10⁻⁷; synaptotagmin 1 (Syt1) p<2.5×10⁻⁵; CUGBP, Elav-like family member 4 (Celf4), p<2.5×10⁻⁴. These genes are promising candidate therapeutic targets for SMA.

It is expected that the inhibition and/or downregulation of the vulnerability genes, or the activation or overexpression of the resistance genes will prevent or slow motor neuron death in SMA.

### DOCUMENTS

Bolger et al., (1993) A family of human phosphodiesterases homologous to the duce learning and memory gene product of Drosophila melanogaster are potential targets for antidepressant drugs. Mold. Cell Biol., 13: 6558-6571.
Dickson et al., (2008) A Negatively Acting Bifunctional RNA Increases Survival Motor Neuron Both In Vitro and In Vivo, Hum Gene Ther. 2008 November; 19(11): 1307-1315.
Keravis, Thérèse and Lugnier, Claire, Cyclic nucleotide phosphodiesterase (PDE) isozymes as targets of the intracellular signalling network: benefits of PDE inhibitors in various diseases and perspectives for future therapeutic developments, British Journal of Pharmacology (2012) 165 1288-1305 Vandeput F, Wolda S L, Krall J, Hambleton R, Uher L, McCaw K N et al. (2007). Cyclic nucleotide phosphodiesterase PDE1C1 in human cardiac myocytes. J Biol Chem 282: 32749-32757.
Le et al., “SMNDelta7, the major product of the centromeric survival motor neuron (SMN2) gene, extends survival in mice with spinal muscular atrophy and associates with full-length SMN.” Hum. Mol. Genet. 14(6):845-57, 2005.
Li, L., et al., (2006) Phosphatidylinositol phosphates as co-activators of Ca2+binding to C2 domains of synaptotagmin 1 J. Biol. Chem. Vol. 281, pp 15845-15852.
Nagel D J, Aizawa T, Jeon K I, Liu W, Mohan A, Wei H et al. (2006). Role of nuclear Ca2+/calmodulin-stimulated phosphodiesterase 1A in vascular smooth muscle cell growth and survival. Circ Res 98: 777-784.
Singh G, Charlet-B N, Han J, Cooper TA. (2004) ETR-3 and CELF4 protein domains required for RNA binding and splicing activity in vivo. Nucleic Acids Res. 18; 32(3):1232-41.
Thomas, N. K., et al., (2001) (S)-3,4-DCPG, a potent and selective mGlu8a receptor agonist, activates metabotropic glutamate receptors on primary afferent terminals in the neonatal rat spinal cord, Neurophamacology, Vol 40, p. 311-318.
U.S. Patent Application Publication No 2010/0298324
U.S. Patent Application Publication No 2011/0144167
U.S. Patent Application Publication No. 2008/0182811
U.S. Patent Application Publication No. 2010/0016434
U.S. Patent Application Publication No. 2011/0039895
U.S. Patent Application Publication No. 2011/0076347
U.S. Patent Application Publication No. 2011/0111058
U.S. Patent Application Publication No. 2012/0121720
U.S. Pat. No. 6,812,239
U.S. Pat. No. 7,608,621

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for treating or ameliorating an effect of proximal spinal muscular atrophy (SMA) comprising administering to a subject in need thereof a modulator of a gene selected from the group consisting of phosphodiesterase 1c (Pde1c), Calbindin 2 (Calb2), Egl nine homolog 3 (Egl3), Metabotropic glutamate receptor 8 (mGluR8), Synaptotagmin 1 (Syt1), CUGBP, Elav-like family member 4 (Celf4), and combinations thereof in an amount effective to treat or ameliorate an effect of SMA.

2. The method according to claim 1, wherein the modulator is an inhibitor of a gene selected from the group consisting of Pde1c, Calb2, Egl3, and combinations thereof.

3. The method according to claim 2, wherein the inhibitor of Pde1c is selected from the group consisting of zaprinast, 8-methoxymethyl-1-methyl-3-(2-methylpropyl)xanthine (8MM-IBMX), vinpocetine, 3-isobutyl-1-methylxanthine (IBMX), SCH51866, Compound 30, Compound 31, nimodipine, IC86340, IC295, IC224, dioclein, KS505a, DIF-1, EGTA, trifluoroperazine, W7, sildenafil, vardenafil, amantadine, deprenyl, ginsenoids, theophylline, HFV-1017, ITI-214, K-259-2, KS-501, KS-505, KS-619-1, Sch-45752, Sch-59498, CV-159, and derivatives of 1-methyl-3-isobutylxanthine.

4. The method according to claim 2, wherein the inhibitor of Calb2 is selected from the group consisting of to cyclosporin A and a dodecapeptide of the sequence ISSIKEKYPSHS (SEQ ID NO. 1).

5. The method according to claim 2, wherein the inhibitor of Egl3 is selected from the group consisting of antianaemic siRNA therapy, iron chelators, dimethyloxaloglycine (DMOG), synthetic 2-oxogluturate antagonists, iron-displacing metals, malonic acid, 3-nitroproprionic acid, theonyl trifluoracetone, 2-oxoglutarate analogs, benzimidazol-4-ylcarboxamide derivatives, pyrimidinedione N-substituted glycine derivatives, pyridazinedione N-substituted glycine derivatives, and 4-ox-2-thioxo-1,2,3,4-tetrahydro-7-quinazolinecarboxamide derivatives.

6. The method according to claim 1, wherein the modulator is an activator of a gene selected from the group consisting of mGluR8, Syt1, Celf4, and combinations thereof.

7. The method according to claim 6, wherein the activator of mGluR8 is selected from the group consisting of L-glutamic acid, cysteine, (S)-3,4-dicarboxyphenylglycine ((S)-3,4-DCPG), (RS)-4-phosphonophenylglycine, L-serine-O-phosphate, and L-2-amino-4 phosphonobutyrate.

8. The method according to claim 6, wherein the activator of Syt1 is a phosphatidylinositol polyphosphate.

9. The method according to claim 6, wherein the activator of Celf4 is selected from the group consisting of CELF4 Δ5.1, CELF4 Δ5.2, CELF4 (+48), CELF4 Δ3.1, CELF4 Δ3.2, CELF4 Δ3.3, CELF4 Δ3.4, CELF4.24, CELF4 DD1, CELF4 DD2, and CELF4 DD3.

10. The method according to claim 1 further comprising co-administering to the subject a modulator of survival motor neuron (SMN) expression.

11. The method according to claim 10, wherein the modulator of SMN expression causes an increase in SMN expression.

12. The method according to claim 11, wherein the modulator of SMN expression is selected from the group consisting of a wild type SMN-1 gene for use in gene therapy, a small molecule, and an antisense oligonucleotide.

13. The method according to claim 12, wherein the small molecule modulator of SMN expression is selected from the group consisting of indoprofen, prolactin, phenylbutyrate, and trichostatin A.

14. The method according to claim 12, wherein the antisense oligonucleotide is exon8-hnRNPA1.

15. The method according to claim 1, wherein the subject is a human.

16. A method for preventing or slowing motor neuron death in a subject having proximal spinal muscular atrophy (SMA) comprising administering to the subject a modulator of a gene selected from the group consisting of Pde1c, Calb2, Egl3, mGluR8, Syt1, Celf4, and combinations thereof in an amount effective to prevent or slow motor neuron death in the subject.

17. The method according to claim 16, wherein the modulator is an inhibitor of a gene selected from the group consisting of Pde1c, Calb2, Egl3, and combinations thereof.

18. The method according to claim 17, wherein the inhibitor of Pde1c is selected from the group consisting of zaprinast, 8-methoxymethyl-1-methyl-3-(2-methylpropyl)xanthine (8MM-IBMX), vinpocetine, 3-isobutyl-1-methylxanthine (IBMX), SCH51866, Compound 30, Compound 31, nimodipine, IC86340, IC295, IC224, dioclein, KS505a, DIF-1, EGTA, trifluoroperazine, W7, sildenafil, vardenafil, amantadine, deprenyl, ginsenoids, theophylline, HFV-1017, ITI-214, K-259-2, KS-501, KS-505, KS-619-1, Sch-45752, Sch-59498, CV-159, and derivatives of 1-methyl-3-isobutylxanthine.

19. The method according to claim 17, wherein the inhibitor of Calb2 is selected from the group consisting of to cyclosporin A and a dodecapeptide of the sequence ISSIKEKYPSHS (SEQ ID NO. 1).

20. The method according to claim 17, wherein the inhibitor of Egl3 is selected from the group consisting of antianaemic siRNA therapy, iron chelators, dimethyloxaloglycine (DMOG), synthetic 2-oxogluturate antagonists, iron-displacing metals, malonic acid, 3-nitroproprionic acid, theonyl trifluoracetone, 2-oxoglutarate analogs, benzimidazol-4-ylcarboxamide derivatives, pyrimidinedione N-substituted glycine derivatives, pyridazinedione N-substituted glycine derivatives, and 4-ox-2-thioxo-1,2,3,4-tetrahydro-7-quinazolinecarboxamide derivatives.

21. The method according to claim 16, wherein the modulator is an activator of a gene selected from the group consisting of mGluR8, Syt1, Celf4, and combinations thereof.

22. The method according to claim 21, wherein the activator of mGluR8 is selected from the group consisting of L-glutamic acid, cysteine, (S)-3,4-dicarboxyphenylglycine ((S)-3,4-DCPG), (RS)-4-phosphonophenylglycine, L-serine-O-phosphate, and L-2-amino-4 phosphonobutyrate.

23. The method according to claim 21, wherein the activator of Syt1 is a phosphatidylinositol polyphosphate.

24. The method according to claim 21, wherein the activator of Celf4 is selected from the group consisting of CELF4 Δ5.1, CELF4 Δ5.2, CELF4 (+48), CELF4 Δ3.1, CELF4 Δ3.2, CELF4 Δ3.3, CELF4 Δ3.4, CELF4.24, CELF4 DD1, CELF4 DD2, and CELF4 DD3.

25. The method according to claim 16 further comprising co-administering to the subject a modulator of survival motor neuron (SMN) expression.

26. The method according to claim 25, wherein the modulator of SMN expression causes an increase in SMN expression.

27. The method according to claim 26, wherein the modulator of SMN expression is selected from the group consisting of a wild type SMN-1 gene for use in gene therapy, a small molecule, and an antisense oligonucleotide.

28. The method according to claim 16, wherein the subject is a human.

29. A pharmaceutical composition for treating or ameliorating an effect of proximal spinal muscular atrophy (SMA) in a subject in need thereof, the pharmaceutical composition comprising a pharmaceutically acceptable carrier or diluent and an amount of a modulator of a gene selected from the group consisting of Pde1c, Calb2, Egl3, mGluR8, Syt1, Celf4, and combinations thereof, which amount is effective to treat or ameliorate an effect of SMA in the subject.

30. The pharmaceutical composition according to claim 29, wherein the modulator is an inhibitor of a gene selected from the group consisting of Pde1c, Calb2, Egl3, and combinations thereof.

31. The pharmaceutical composition according to claim 30, wherein the inhibitor of Pde1c is selected from the group consisting of zaprinast, 8-methoxymethyl-1-methyl-3-(2-methylpropyl)xanthine (8MM-IBMX), vinpocetine, 3-isobutyl-1-methylxanthine (IBMX), SCH51866, Compound 30, Compound 31, nimodipine, IC86340, IC295, IC224, dioclein, KS505a, DIF-1, EGTA, trifluoroperazine, W7, sildenafil, vardenafil, amantadine, deprenyl, ginsenoids, theophylline, HFV-1017, ITI-214, K-259-2, KS-501, KS-505, KS-619-1, Sch-45752, Sch-59498, CV-159, and derivatives of 1-methyl-3-isobutylxanthine.

32. The pharmaceutical composition according to claim 30, wherein the inhibitor of Calb2 is selected from the group consisting of to cyclosporin A and a dodecapeptide of the sequence ISSIKEKYPSHS (SEQ ID NO. 1).

33. The pharmaceutical composition according to claim 30, wherein the inhibitor of Egl3 is selected from the group consisting of antianaemic siRNA therapy, iron chelators, dimethyloxaloglycine (DMOG), synthetic 2-oxogluturate antagonists, iron-displacing metals, malonic acid, 3-nitroproprionic acid, theonyl trifluoracetone, 2-oxoglutarate analogs, benzimidazol-4-ylcarboxamide derivatives, pyrimidinedione N-substituted glycine derivatives, pyridazinedione N-substituted glycine derivatives, and 4-ox-2-thioxo-1,2,3,4-tetrahydro-7-quinazolinecarboxamide derivatives.

34. The pharmaceutical composition according to claim 29, wherein the modulator is an activator of a gene selected from the group consisting of mGluR8, Syt1, Celf4, and combinations thereof.

35. The pharmaceutical composition according to claim 34, wherein the activator of mGluR8 is selected from the group consisting of L-glutamic acid, cysteine, (S)-3,4-dicarboxyphenylglycine ((S)-3,4-DCPG), (RS)-4-phosphonophenylglycine, L-serine-O-phosphate, and L-2-amino-4 phosphonobutyrate.

36. The pharmaceutical composition according to claim 34, wherein the activator of Syt1 is a phosphatidylinositol polyphosphate.

37. The pharmaceutical composition according to claim 34, wherein the activator of Celf4 is selected from the group consisting of CELF4 Δ5.1, CELF4 Δ5.2, CELF4 (+48), CELF4 Δ3.1, CELF4 Δ3.2, CELF4 Δ3.3, CELF4 Δ3.4, CELF4.24, CELF4 DD1, CELF4 DD2, and CELF4 DD3.

38. The pharmaceutical composition according to claim 29 further comprising a modulator of survival motor neuron (SMN) expression.

39. The pharmaceutical composition according to claim 38 wherein the modulator of SMN expression causes an increase in SMN expression.

40. The pharmaceutical composition according to claim 39, wherein the modulator of SMN expression is selected from the group consisting of a wild type SMN-1 gene for use in gene therapy, a small molecule, and an antisense oligonucleotide.

41. A method for preventing or slowing motor neuron death comprising contacting a motor neuron with a modulator of a gene selected from the group consisting of Pde1c, Calb2, Egl3, mGluR8, Syt1, Celf4, and combinations thereof in an amount effective to prevent or slow motor neuron death.

42. The method according to claim 41, wherein the modulator is an inhibitor of a gene selected from the group consisting of Pde1c, Calb2, Egl3, and combinations thereof.

43. The method according to claim 42, wherein the inhibitor of Pde1c is selected from the group consisting of zaprinast, 8-methoxymethyl-1-methyl-3-(2-methylpropyl)xanthine (8MM-IBMX), vinpocetine, 3-isobutyl-1-methylxanthine (IBMX), SCH51866, Compound 30, Compound 31, nimodipine, IC86340, IC295, IC224, dioclein, KS505a, DIF-1, EGTA, trifluoroperazine, W7, sildenafil, vardenafil, amantadine, deprenyl, ginsenoids, theophylline, HFV-1017, ITI-214, K-259-2, KS-501, KS-505, KS-619-1, Sch-45752, Sch-59498, CV-159, and derivatives of 1-methyl-3-isobutylxanthine.

44. The method according to claim 42, wherein the inhibitor of Calb2 is selected from the group consisting of to cyclosporin A and a dodecapeptide of the sequence ISSIKEKYPSHS (SEQ ID NO. 1).

45. The method according to claim 42, wherein the inhibitor of Egl3 is selected from the group consisting of antianaemic siRNA therapy, iron chelators, dimethyloxaloglycine (DMOG), synthetic 2-oxogluturate antagonists, iron-displacing metals, malonic acid, 3-nitroproprionic acid, theonyl trifluoracetone, 2-oxoglutarate analogs, benzimidazol-4-ylcarboxamide derivatives, pyrimidinedione N-substituted glycine derivatives, pyridazinedione N-substituted glycine derivatives, and 4-ox-2-thioxo-1,2,3,4-tetrahydro-7-quinazolinecarboxamide derivatives.

46. The method according to claim 41, wherein the modulator is an activator of a gene selected from the group consisting of mGluR8, Syt1, Celf4, and combinations thereof.

47. The method according to claim 46, wherein the activator of mGluR8 is selected from the group consisting of L-glutamic acid, cysteine, (S)-3,4-dicarboxyphenylglycine ((S)-3,4-DCPG), (RS)-4-phosphonophenylglycine, L-serine-O-phosphate, and L-2-amino-4 phosphonobutyrate.

48. The method according to claim 46, wherein the activator of Syt1 is a phosphatidylinositol polyphosphate.

49. The method according to claim 46, wherein the activator of Celf4 is selected from the group consisting of CELF4 Δ5.1, CELF4 E5.2, CELF4 (+48), CELF4 Δ3.1, CELF4 Δ3.2, CELF4 Δ3.3, CELF4 Δ3.4, CELF4.24, CELF4 DD1, CELF4 DD2, and CELF4 DD3.

50. The method according to claim 41 further comprising contacting the motor neuron with a modulator of survival motor neuron (SMN) expression.

51. The method according to claim 50, wherein the modulator of SMN expression causes an increase in SMN expression.

52. The method according to claim 51, wherein the modulator of SMN expression is selected from the group consisting of a wild type SMN-1 gene for use in gene therapy, a small molecule, and an antisense oligonucleotide.

53. The method according to claim 41, wherein the motor neuron is a human motor neuron.
